(19) United States
(12) Patent Application Publication
(10) Pub. No.: US 2020/0221959 A1
(43) Pub. Date: Jul. 16, 2020

(54) VITAL SIGN MEASUREMENT DEVICE, BLOOD PRESSURE MEASUREMENT DEVICE, APPARATUS, VITAL SIGN MEASUREMENT METHOD, AND BLOOD PRESSURE MEASUREMENT METHOD

(71) Applicants: OMRON CORPORATION, Kyoto-shi (JP); OMRON HEALTHCARE CO., LTD., Muko-shi (JP)

(72) Inventors: Keigo KAMADA, Tokyo (JP); Keisuke SAITO, Suita-shi (JP); Hisashi OZAWA, Kyoto-shi (JP); Yasuhiro KAWABATA, Kyoto (JP)

(73) Assignees: OMRON CORPORATION, Kyoto-shi (JP); OMRON HEALTHCARE CO., LTD., Muko-shi (JP)

(21) Appl. No.: 16/836,360

(22) Filed: Mar. 31, 2020

Related U.S. Application Data
(63) Continuation of application No. PCT/JP2018/034642, filed on Sep. 19, 2018.

(30) Foreign Application Priority Data
Oct. 12, 2017 (JP) 2017-198498

(51) Int. Cl. A61B 5/0245; A61B 5/024; A61B 5/021; A61B 5/022

(52) U.S. Cl. CPC A61B 5/0245; A61B 5/02438; A61B 2562/182; A61B 5/02233; A61B 5/02225; A61B 5/02125

(57) ABSTRACT

A vital sign measurement device includes a belt to be worn around an upper limb of a living body, and a transmission and reception unit provided to the belt, capable of transmitting and receiving radio waves. The transmission and reception unit includes transmission and reception antenna units. The transmission antenna unit emits radio waves respectively toward an artery of the upper limb and a heart. The reception antenna unit receives radio waves respectively reflected by the artery and/or a tissue being displaced in accordance with a pulse wave of the artery and by the heart and/or a tissue being displaced in accordance a heartbeat of the heart. The vital sign measurement device includes a vital sign detection unit that acquires a pulse wave signal representing the pulse wave of the artery and a heartbeat signal representing the heartbeat of the heart based on the outputs from the reception antenna unit.

### CROSS-REFERENCE TO RELATED APPLICATION

This is a continuation application of International Application No. PCT/JP2018/034642, with an International filing date of Sep. 19, 2018, which claims priority of Japanese Patent Application No. 2017-198498 filed on Oct. 12, 2017, the entire content of which is hereby incorporated by reference.

### TECHNICAL FIELD

The present invention relates to a vital sign measurement device, and more particularly, to a vital sign measurement device that measures a pulse wave of an artery and a heartbeat of a heart of a living body. In addition, the present invention relates to a blood pressure measurement device and an apparatus including such a vital sign measurement device. The present invention further relates to a vital sign measurement method for measuring a pulse wave of an artery and a heartbeat of a heart of a living body. The present invention also relates to a blood pressure measurement method including such a vital sign measurement method.

### BACKGROUND ART

Patent Literature 1 (JP 2012-139342 A) discloses a conventional example of this type of vital sign measurement device. The device includes a plurality of electrodes mounted or attached to portions of the living body sandwiching the heart. Electrocardiographic waves are output based on signals generated by the plurality of electrodes. Furthermore, a pulse wave sensor (a cuff for example) that is worn around an upper arm of the living body and detects the pulse waves transmitted in the artery is provided. The pulse transit time is detected based on a time difference between a point when an R wave included in the electrocardiographic waves is generated and a timing when the pulse wave is detected by the pulse wave sensor.

Furthermore, Patent Literature 2 (JP 2016-150065 A) discloses the following technique. Specifically, two microwave sensors are arranged below a mattress so as to be separated from each other in a horizontal direction. One of the microwave sensors irradiates a trunk part of a subject lying on the mattress with microwaves. As a result, a sensor signal is received from the trunk part. Furthermore, the other microwave sensor irradiates a distal portion of the subject with microwaves. As a result, a sensor signal is received from the distal portion.

### SUMMARY OF INVENTION

The device described in Patent Literature 1 requires a plurality of electrodes to be mounted or attached to portions of a living body sandwiching the heart. Thus a cumbersome process of attaching them to the living body is required. On top of that, a large physical burden is imposed on the living body (subject) for maintaining the attached state.

On the other hand, the device described in Patent Literature 2 is free of the cumbersome attaching process. Still, a large physical burden is imposed on the subject because he or she has to lie on the mattress.

In view of this, an object of the present invention is to provide a vital sign measurement device that measures a pulse wave of an artery and a heartbeat of the heart of a living body, while imposing a small physical burden on the living body during the measurement. Another object of the present invention is to provide a blood pressure measurement device and an apparatus including such a vital sign measurement device. A further object of the present invention is to provide a vital sign measurement method of measuring a pulse wave of an artery and a heartbeat of the heart of a living body by using such a vital sign measurement device. A further object of the present invention is to provide a blood pressure measurement method including such a vital sign measurement method.

In order to achieve the object, a vital sign measurement device of the present disclosure is a vital sign measurement device that measures a pulse wave of an artery and a heartbeat of a heart of a living body, the vital sign measurement device comprising:

a belt to be worn around an upper limb part of the living body; and

a transmission and reception unit that is capable of transmitting and receiving radio waves, the transmission and reception unit being provided at a portion of the belt to face both an artery running in the upper limb part and the heart when the living body takes a predetermined recommended measurement posture in a worn state of the belt being worn around the upper limb part, wherein

the transmission and reception unit includes:

a transmission antenna unit that emits radio waves to each of the artery in the upper limb part and the heart; and

a reception antenna unit that receives radio waves reflected by the artery in the upper limb part and/or a tissue being displaced in accordance with a pulse wave of the artery and by the heart and/or a tissue being displaced in accordance with the heartbeat of the heart, and

the vital sign measurement device further comprises a vital sign detection unit that acquires a pulse wave signal representing the pulse wave of the artery in the upper limb part and a heartbeat signal representing the heartbeat of the heart based on an output from the reception antenna unit.

As used herein, the “upper limb part” includes the upper arms, the forearms, the hands, and the fingers.

The portion of the belt at which the transmission and reception unit is mounted is set in advance as a portion facing both the artery running in the upper limb part and the heart, when the living body takes a predetermined “recommended measurement posture” in a state where the belt is worn around the upper limb part. The term “facing” may indicate any state where the radio waves can be transmitted and received to and from each other, between the transmission and reception unit and the upper limb part, and between the transmission and reception unit and the heart. Thus, facing each other indirectly with clothes and the like provided therebetween is included.

As the “recommended measurement posture”, a posture where the artery in the upper limb part and the heart are (almost) at the same height, with respect to the direction of gravitational acceleration or the like, is recommended. For example, when the upper limb part is an upper arm, a posture with the upper arm extending along a side of the trunk may be employed. Alternatively, when the upper limb part is a wrist, the following “recommended measurement posture” may be employed in a state where the living body stands straight. Specifically, a subject raises his or her forearm so that the forearm diagonally extends (hand up, elbow down) in front of and while overlapping with the trunk. The wrist is maintained at the same height level as the heart. The palm side surface of the wrist (a part of the outer circumferential surface of the wrist corresponding to the palm) faces the heart. When the upper limb part is the wrist and the living body is lying on his/her back, the posture with the wrist put on the front chest is not recommended.

The “tissue being displaced in accordance with the pulse wave of the artery” of the upper limb part is a portion of the living body that is displaced in accordance with the pulse wave of the artery (causing the expansion and contraction of blood vessels). For example, in a “skin-fatty layer-artery” configuration, a skin of the upper limb part is included. The “tissue being displaced in accordance with the heartbeat of the heart” is a portion of the living body that is displaced in accordance with the heartbeat of the heart.

In another aspect, a blood pressure measurement device of the present disclosure is a blood pressure measurement device that measures blood pressure of a living body, the blood pressure measurement device comprising:

the above vital sign measurement device;

a time difference acquisition unit that acquires as a pulse transit time, a time difference between the pulse wave signal and the heartbeat signal acquired by the vital sign detection unit; and

a first blood pressure calculation unit that calculates a blood pressure value based on the pulse transit time acquired by the time difference acquisition unit by using a predetermined correspondence formula between the pulse transit time and the blood pressure.

In another aspect, an apparatus of the present disclosure is an apparatus comprising the above vital sign measurement device or the above blood pressure measurement device.

In another aspect, a vital sign measurement method of the present disclosure is a vital sign measurement method that measures a pulse wave of an artery and a heartbeat of a heart of a living body by using the above vital sign measurement device, the vital sign measurement method comprising:

wearing the belt around the upper limb part; and

causing the transmission and reception unit to face both an artery running in the upper limb part and the heart by the living body taking a predetermined posture in a worn state of the belt being worn around the upper limb part;

emitting radio waves to each of the artery in the upper limb part and the heart through the transmission antenna unit;

receiving radio waves reflected by the artery in the upper limb part and/or a tissue being displaced in accordance with a pulse wave of the artery and by the heart and/or a tissue being displaced in accordance with the heartbeat of the heart through the reception antenna unit; and

acquiring, by the vital sign detection unit, a pulse wave signal representing the pulse wave of the artery in the upper limb part and a heartbeat signal representing the heartbeat of the heart based on an output from the reception antenna unit.

In another aspect, a blood pressure measurement method of the present disclosure is a blood pressure measurement method that measures blood pressure of a living body, the blood pressure measurement method comprising:

acquiring a pulse wave signal representing the pulse wave of the artery in the upper limb part and a heartbeat signal representing the heartbeat of the heart by executing the above vital sign measurement method;

acquiring, as a pulse transit time, a time difference between the pulse wave signal and the heartbeat signal; and

calculating a blood pressure value based on the acquired pulse transit time by using a predetermined correspondence formula between the pulse transit time and the blood pressure.

### BRIEF DESCRIPTION OF DRAWINGS

The present invention will become more fully understood from the detailed description given hereinbelow and the accompanying drawings which are given by way of illustration only, and thus are not limitative of the present invention, and wherein:

FIG. 1 is a diagram illustrating an application example in which a vital sign measurement device of one embodiment of the present invention is attached to a living body to acquire a vital sign.

FIG. 2 is a perspective view illustrating an external appearance of a wrist-type sphygmomanometer according to one embodiment of the vital sign measurement device and a blood pressure measurement device according to the present invention.

FIG. 3 is a diagram schematically illustrating a cross section orthogonal to a longitudinal direction of a left wrist in a state where the sphygmomanometer is worn on the left wrist.

FIG. 4 is a diagram illustrating a planar layout of an example of a transmission and reception antenna group in a state where the sphygmomanometer is worn on the left wrist.

FIG. 5 is a diagram illustrating a state in which a subject wearing the sphygmomanometer on the left wrist is taking a predetermined recommended measurement posture.

FIG. 6A is a diagram illustrating a cross-sectional structure of the example of the transmission and reception antenna group together with their directivities. FIG. 6B is a diagram illustrating a modification of the cross-sectional structure in FIG. 6A.

FIG. 7A is a diagram illustrating a cross-sectional structure of the transmission and reception antenna group corresponding to FIG. 6A. FIG. 7B illustrates an example of feed points and polarization directions of transmission antennas and reception antennas included in the transmission and reception antenna group illustrated in FIG. 7A as is viewed from the left side (+Z direction). FIG. 7C illustrates an example of feed points and polarization directions of the transmission antennas and the reception antennas illustrated in FIG. 7A as is viewed from the right side (−Z direction).

FIG. 8 is a diagram illustrating an overall block configuration of a control system of the sphygmomanometer.

FIG. 9 is a diagram illustrating a partial and functional block configuration of the control system of the sphygmomanometer.

FIG. 10 is a diagram illustrating a block configuration implemented in the sphygmomanometer by a program for performing an oscillometric method.

FIG. 11 is a diagram illustrating an operation flow when the sphygmomanometer measures the blood pressure through the oscillometric method.

FIG. 12 is a diagram illustrating changes in cuff pressure and pulse wave signal according to the operation flow in FIG. 11.

FIG. 13 is a diagram illustrating waveforms of a pulse wave signal obtained from the left wrist and a heartbeat signal obtained from the heart, and Pulse Transit Time (PTT) obtained from the pulse wave signal and the heartbeat signal.

FIG. 14 is a diagram illustrating an operation flow according to a vital sign measurement method and a blood pressure measurement method according to one embodiment of the present invention, in which the sphygmomanometer acquires PTT and performs the blood pressure measurement (estimation) based on the PTT.

FIG. 15 is a diagram illustrating an example where in the block configuration illustrated in FIG. 9, a frequency f1 of radio waves E1 emitted toward the artery in the left wrist and a frequency f2 of radio waves E2 emitted toward the heart are different from each other.

FIG. 16A is a diagram illustrating an example of another arrangement of the transmission antennas and the reception antennas included in the transmission and reception antenna group in a cross section (ZX plane) corresponding to FIG. 3. FIG. 16B is a diagram illustrating in a cross section (YZ plane) of what is illustrated in FIG. 16A, taken along the longitudinal direction of the left wrist.

FIG. 17A is a diagram illustrating an example of still another arrangement of the transmission antennas and the reception antennas included in the transmission and reception antenna group in a cross section (ZX plane) corresponding to FIG. 3. FIG. 17B is a diagram illustrating what is illustrated in FIG. 17A, in a cross section (YZ plane) taken along the longitudinal direction of the left wrist.

FIG. 18A is a diagram illustrating an example of yet still another arrangement of the transmission antennas and the reception antennas included in the transmission and reception antenna group in a cross section (ZX plane) corresponding to FIG. 3. FIG. 18B is a diagram illustrating what is illustrated in FIG. 18A, in a cross section (YZ plane) taken along the longitudinal direction of the left wrist.

FIG. 19A is a diagram illustrating another example of transmission antennas and reception antennas having the same cross-sectional structure as the cross-sectional structure illustrated in FIG. 7A. FIGS. 19B and 19C illustrate examples of feed points and polarization directions of the transmission antennas and the reception antennas illustrated in FIG. 19A as is viewed from the left side (+Z direction) and the right side (−Z direction), respectively.

FIG. 20A is a diagram illustrating a cross-sectional structure of the transmission antennas and the reception antennas illustrated in FIGS. 18A and 18B. FIGS. 20B and 20C illustrate examples of feed points and polarization directions of the transmission antennas and the reception antennas illustrated in FIG. 20A as is viewed from the left side (+Z direction) and the right side (−Z direction), respectively.

FIG. 21A is a diagram illustrating another example of transmission antennas and reception antennas having the same cross-sectional structure as the cross-sectional structure illustrated in FIG. 20A. FIGS. 21B and 21C illustrate examples of feed points and polarization directions of the transmission antennas and the reception antennas illustrated in FIG. 21A as is viewed from the left side (+Z direction) and the right side (−Z direction), respectively.

FIG. 22A is a diagram illustrating another example of transmission antennas and reception antennas having the same cross-sectional structure as the cross-sectional structure illustrated in FIG. 7A. FIGS. 22B and 22C illustrate examples of feed points and polarization directions of the transmission antennas and the reception antennas illustrated in FIG. 22A as is viewed from the left side (+Z direction) and the right side (−Z direction), respectively.

FIG. 23A is a diagram illustrating another example of transmission antennas and reception antennas having the same cross-sectional structure as the cross-sectional structure illustrated in FIG. 20A. FIGS. 23B and 23C illustrate examples of feeding points and polarization directions of the transmission antennas and the reception antennas illustrated in FIG. 23A as is viewed from the left side (+Z direction) and the right side (−Z direction), respectively.

FIG. 24A is a diagram illustrating another example of transmission antennas and reception antennas having the same cross-sectional structure as the cross-sectional structure illustrated in FIG. 7A. FIGS. 24B and 24C show feeding points and polarization directions of the transmission antennas and the reception antennas when the device of FIG. 24A is viewed from the left side (+Z direction) and the right side (−Z direction), respectively.

FIG. 25A is a diagram illustrating another example of transmission antennas and reception antennas having the same cross-sectional structure as the cross-sectional structure illustrated in FIG. 7A. FIGS. 25B and 25C illustrate examples of feed points and polarization directions of the transmission antennas and the reception antennas illustrated in FIG. 25A as is viewed from the left side (+Z direction) and the right side (−Z direction), respectively.

FIG. 26A is a diagram illustrating another example of transmission antennas and reception antennas having the same cross-sectional structure as the cross-sectional structure illustrated in FIG. 20A. FIGS. 26B and 26C illustrate examples of feed points and polarization directions of the transmission antennas and the reception antennas illustrated in FIG. 26A as is viewed from the left side (+Z direction) and the right side (−Z direction), respectively.

FIG. 27A is a diagram illustrating another example of transmission antennas and reception antennas having the same cross-sectional structure as the cross-sectional structure illustrated in FIG. 20A. FIGS. 27B and 27C show feeding points and polarization directions of the transmission antennas and the reception antennas when the device of FIG. 27A is viewed from the left side (+Z direction) and the right side (−Z direction), respectively.

FIG. 28A is a diagram illustrating another example of transmission antennas and reception antennas having the same cross-sectional structure as the cross-sectional structure illustrated in FIG. 7A. FIGS. 28B and 28C show feeding points and polarization directions of the transmission antennas and the reception antennas when the device of FIG. 28A is viewed from the left side (+Z direction) and the right side (−Z direction), respectively.

FIG. 29A is a diagram illustrating another example of transmission antennas and reception antennas having the same cross-sectional structure as the cross-sectional structure illustrated in FIG. 20A. FIGS. 29B and 29C show feeding points and polarization directions of the transmission antennas and the reception antennas when the device of FIG. 29A is viewed from the left side (+Z direction) and the right side (−Z direction), respectively.

FIG. 30A is a diagram illustrating a planar layout of another example of a transmission and reception antenna group in a state where the sphygmomanometer is worn on the left wrist. FIG. 30B is a schematic cross-sectional view of what is illustrated in FIG. 30A, taken along the longitudinal direction (Y direction) of left wrist.

FIG. 31A is a diagram illustrating a cross-sectional structure of the transmission and reception antenna group illustrated in FIGS. 30A and 30B. FIG. 31B is a diagram illustrating a modification of the cross-sectional structure in FIG. 31A.

FIG. 32 is a diagram illustrating a directivity of a dipole antenna.

FIG. 33 is a diagram illustrating a polarization direction obtained by a dipole antenna.

FIG. 34A illustrates directivities and polarization directions of the transmission antenna and the reception antennas illustrated in FIG. 30A, in a plane (XY plane) corresponding to FIG. 30A. FIG. 34B is a diagram illustrating the directivities of the transmission antenna and the reception antennas in a plane (YZ plane) corresponding to FIG. 30B.

FIG. 35 is a diagram illustrating a partial and functional block configuration of a control system in a case where the sphygmomanometer includes the transmission antenna and the reception antennas illustrated in FIGS. 30A and 30B.

FIG. 36 is a diagram illustrating an example, in the block configuration of the control system illustrated in FIG. 35, where a frequency component (frequency f1) of radio waves E1′ reflected by the artery in the left wrist and received through the first reception antenna and a frequency component (frequency f2) of radio waves E2′ reflected by the heart and received through the second reception antenna are different from each other.

FIG. 37A is a diagram corresponding to FIG. 30A and illustrating an example of another arrangement of the transmission antenna and the reception antennas included in the transmission and reception antenna group illustrated in FIG. 30A. FIG. 37B is a diagram illustrating what is illustrated in FIG. 37A, in a cross section (YZ plane) taken along the longitudinal direction of the left wrist.

FIG. 38A is a diagram corresponding to FIG. 30A and illustrating another example of another arrangement of the transmission antenna and the reception antennas included in the transmission and reception antenna group illustrated in FIG. 30A. FIG. 38B is a diagram illustrating what is illustrated in FIG. 38A, in a cross section (YZ plane) taken along the longitudinal direction of the left wrist.

FIG. 39A is a diagram corresponding to FIG. 30A and illustrating another example of another arrangement of the transmission antenna and the reception antennas included in the transmission and reception antenna group illustrated in FIG. 30A. FIG. 39B is a diagram illustrating what is illustrated in FIG. 39A, in a cross section (YZ plane) taken along the longitudinal direction of the left wrist.

### DESCRIPTION OF EMBODIMENTS

Hereinafter, embodiments of the present invention will be described in detail with reference to the drawings.

### Application Example

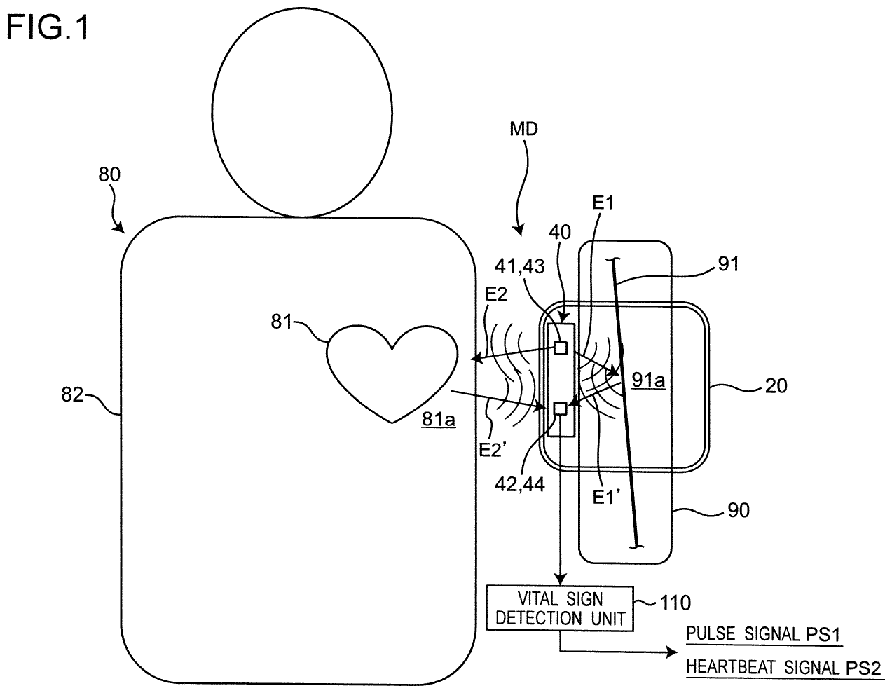

FIG. 1 illustrates an application example in which a vital sign measurement device (denoted with a sign MD) of one embodiment of the present invention is attached to a living body 80 to acquire vital signs. Here, the living body 80 has a trunk 82 including a heart 81 and an upper limb part 90 in which an artery 91 extending from the heart 81 runs. In FIG. 1, the trunk 82 and the upper limb part 90 are each represented by a rounded square, and the heart 81 is schematically represented by a heart mark. The upper limb part 90 may be any part from the shoulder to the fingertip, such as an upper arm, a forearm, a hand, or a finger.

The vital sign measurement device MD is a device that measures the pulse wave of the artery 91 and the heartbeat of the heart 81 of the living body 80, and includes a belt 20 that is worn around the upper limb part 90 of the living body 80 and a transmission and reception unit 40 that is provided to the belt 20 and can transmit and receive radio waves. The transmission and reception unit 40 is provided at a portion in the belt 20 facing both the artery 91 running in the upper limb part 90 and the heart 81, when the living body 80 takes a predetermined recommended measurement posture in a worn state where the belt 20 is worn around the upper limb part 90. Here, when the upper limb part 90 is an upper arm, for example, a posture with the upper arm extending along a side of the trunk 82 is employed as the “predetermined recommended measurement posture”. Furthermore, the term “facing” may indicate any state where the radio waves can be transmitted and received to and from each other, between the transmission and reception unit 40 and the upper limb part 90, and between the transmission and reception unit 40 and the heart 81. Thus, facing each other indirectly with clothes and the like provided therebetween is included.

The transmission and reception unit 40 includes transmission antennas 41 and 43 and reception antennas 42 and 44. The transmission antennas 41 and 43 serve as a transmission antenna unit that emits radio waves E1 and E2 respectively toward the artery 91 in the upper limb part 90 and the heart 81. The reception antennas 42 and 44 serve as a reception antenna unit that receive radio waves E1′ and E2′ respectively reflected by the artery 91 in the upper limb part 90 and/or a tissue 91a being displaced in accordance with a pulse wave of the artery 91 and by the heart 81 and/or a tissue 81a being displaced in accordance a heartbeat of the heart 81. Here, the “tissue 91a being displaced in accordance with the pulse wave of the artery 91” of the upper limb part 90 is a portion of the living body 80 that is displaced in accordance with the pulse wave of the artery 91 (causing the expansion and contraction of blood vessels). For example, in a “skin-fatty layer-artery” configuration, a skin of the upper limb part 90 is included. The “tissue 81a being displaced in accordance with the heartbeat of the heart 81” is a portion of the living body 80 that is displaced in accordance with the heartbeat of the heart 81.

The vital sign measurement device MD further includes a vital sign detection unit 110 that acquires a pulse wave signal PS1 representing the pulse wave of the artery 91 in the upper limb part 90 and a heartbeat signal PS2 representing the heartbeat of the heart 81 based on the outputs from the reception antennas 42 and 44. The vital sign detection unit 110 can be formed by a signal processing system including, for example, a Central Processing Unit (CPU). The pulse wave signal PS1 and the heartbeat signal PS2 are, for example, signals having a mountain-like waveform as illustrated in FIG. 13 (the horizontal axis and the vertical axis in FIG. 13 respectively represent time t and signal voltage v).

In this vital sign measurement device MD, the transmission and reception unit 40 faces both the artery 91 running in the upper limb part 90 and the heart 81, when the living body 80 takes the predetermined recommended measurement posture, in the worn state where the belt 20 is worn around the upper limb part 90 of the living body 80 as illustrated in FIG. 1. The transmission antennas 41 and 43 included in the transmission and reception unit 40 emit radio waves E1 and E2 respectively toward the artery 91 of the upper limb part 90 and the heart 81. The reception antennas 42 and 44 included in the transmission and reception unit 40 receive the radio waves E1′ and E2′ reflected by the artery 91 in the upper limb part 90 and/or the tissue 91a being displaced in accordance with a pulse wave of the artery 91 and by the heart 81 and/or the tissue 81a being displaced in accordance with the heartbeat of the heart 81. The vital sign detection unit 110 acquires the pulse wave signal PS1 representing the pulse wave of the artery 91 in the upper limb part 90 and the heartbeat signal PS2 representing the heartbeat of the heart 81 based on the outputs from the reception antennas 42 and 44.

In this manner, in this vital sign measurement device MD, the pulse wave signal PS1 representing a pulse wave of the artery 91 in the upper limb part 90 and the heartbeat signal PS2 representing the heartbeat of the heart 81 are acquired simply with the living body 80 physically wearing the belt 20 wound around the upper limb part 90 and taking the predetermined recommended measurement posture. Thus, for the measurement, no electrode needs to be mounted or attached to portions of the living body 80 surrounding the heart 81. Furthermore, the recommended measurement posture taken by the living body 80 may include a wide variety of postures such as a posture with the upper body erected or a lying posture, and thus a degree of freedom is high. Therefore, the vital sign measurement device MD imposes a small physical burden on the living body 80 for the measurement.

### Configuration Example

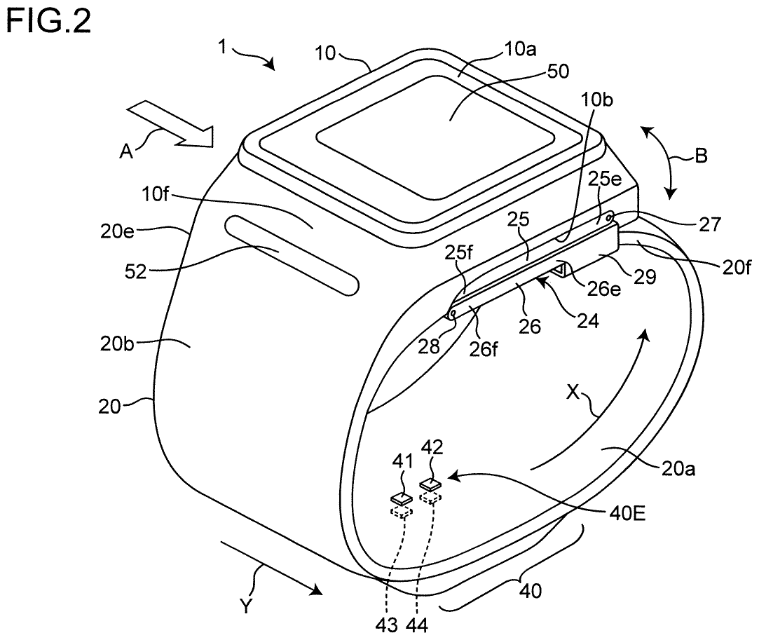

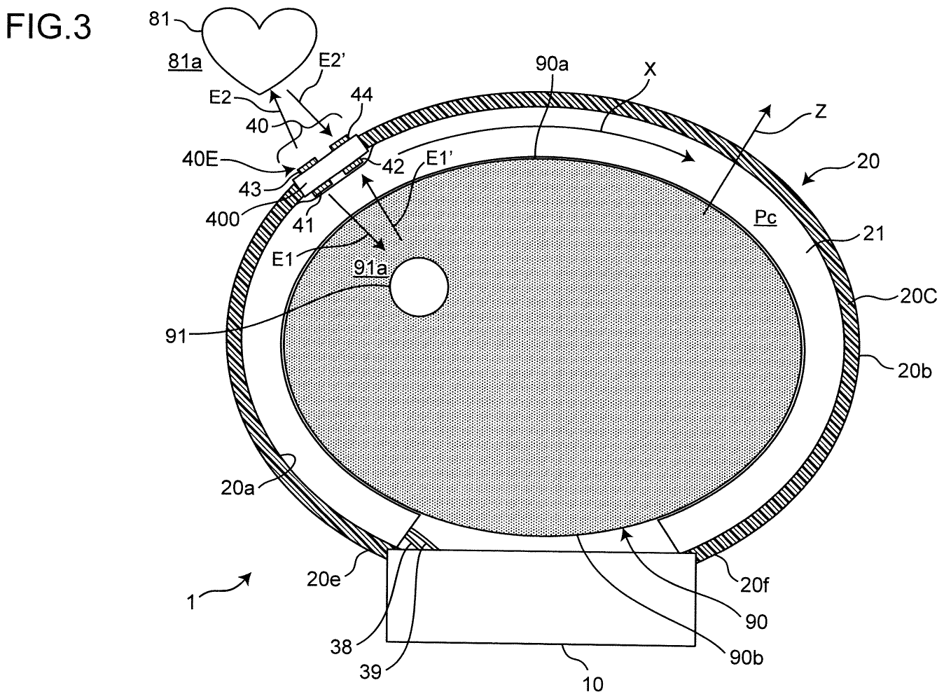

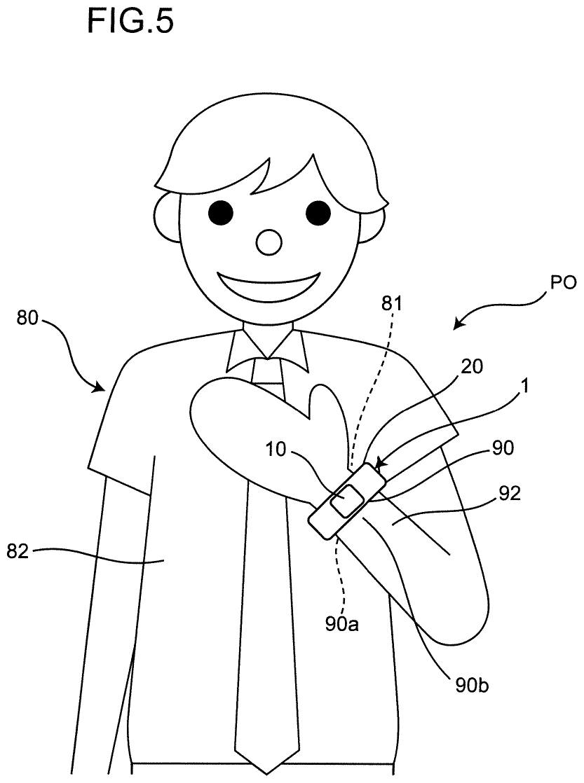

FIG. 2 is a perspective view illustrating an external appearance of a wrist-type sphygmomanometer (whose entirety is indicated by reference numeral 1) which is an embodiment of the vital sign measurement device and the blood pressure measurement device according to an example of the present invention. FIG. 3 is a schematic cross-sectional view taken along a direction orthogonal to the longitudinal direction of a left wrist 90 (denoted with the same reference numeral as the upper limb part 90 in FIG. 1 for the sake of simplicity), in a state where the sphygmomanometer 1 is worn around the left wrist 90 (hereinafter, referred to as a “worn state”) as the upper limb part of a subject 80 (see FIG. 5. denoted with the same reference numeral as the living body 80 for the sake of simplicity). In the following description, the same elements in the drawings are denoted with the same reference numerals, and redundant description will be omitted.

As illustrated in FIGS. 2 and 3, the sphygmomanometer 1 mainly includes the belt 20 worn around the left wrist 90 of the subject 80 as a user, and a main body 10 integrally attached to the belt 20.

As can be seen in FIG. 2, the belt 20 has an elongated band shape so as to surround the left wrist 90 along the circumferential direction, and has an inner circumferential surface 20a to be in contact with the left wrist 90, and an outer circumferential surface 20b on the side opposite to this inner circumferential surface 20a. The dimension (width dimension) of the belt 20 in a width direction Y is set to be about 30 mm in this example.

The main body 10 is integrally provided to one end portion 20e of the belt 20 in the circumferential direction by integral molding in this example. The belt 20 and the main body 10 may be formed separately, and the main body 10 may be integrally attached to the belt 20 using an engaging member (for example, a hinge). In this example, the portion where the main body 10 is arranged is expected to correspond to a back side surface (surface of back side) 90b of the left wrist 90 in the worn state (see FIG. 3). FIG. 3 illustrates the artery (radial artery, in this example) 91 that runs in the vicinity of the palm side surface (surface of palm-side) 90a as an outer surface, in the left wrist 90. The artery may include the ulnar artery.

As can be seen in FIG. 2, the main body 10 has a three-dimensional shape having a thickness in a direction orthogonal to the outer circumferential surface 20b of the belt 20. The main body 10 is formed to be small and thin so as not to disturb the daily activities of the subject 80. In this example, the main body 10 has a quadrangular frustum-shaped outline projecting outward from the belt 20.

On the top surface (the surface farthest from the left wrist 90) 10a of the main body 10, a display 50 forming a display screen is provided. An operation unit 52 with which an instruction is input from the subject 80 is provided along a side surface 10f of the main body 10 (a left near side surface in FIG. 2).

The transmission and reception unit 40 is integrally provided to a portion of the belt 20 between the one end portion 20e and another end portion 20f in the circumferential direction. In this example, the transmission and reception unit 40 is equipped with four transmission and reception antennas 41 to 44 (referred to as a “transmission and reception antenna group” and denoted with a reference numeral 40E). The first transmission antenna 41 and the first reception antenna 42 are arranged on the inner circumferential surface 20a side of the belt 20 while being separated from each other in the longitudinal direction X of the belt 20. The second transmission antenna 43 and the second reception antenna 44 are arranged on the outer circumferential surface 20b side of the belt 20 while being separated from each other in the longitudinal direction X of the belt 20, and at positions respectively corresponding to the transmission antenna 41 and the reception antenna 42 described above (the transmission and reception antenna group 40E will be described later in detail.). In this example, the portion where the transmission and reception antenna group 40E is arranged is expected to correspond to the radial artery 91 of the left wrist 90 in the worn state (see FIG. 3), in the longitudinal direction X of the belt 20. A pressing cuff 21 (described later) provided along the inner circumferential surface 20a of the belt 20 is omitted in FIG. 2 for easy understanding.

As illustrated in FIG. 2, a bottom surface (the surface closest to the left wrist 90) 10b of the main body 10 and the end portion 20f of the belt 20 are connected to each other via a three-fold buckle 24. The buckle 24 includes a first plate member 25 disposed on the outer circumference side and a second plate member 26 disposed on the inner circumference side. The first plate member 25 has one end portion 25e rotatably attached to the main body 10 via a connecting rod 27 extending along the width direction Y. The first plate member 25 has the other end portion 25f rotatably attached to one end portion 26f of the second plate member 26 via a connecting rod 28 extending along the width direction Y. The other end portion 26e of the second plate member 26 is fixed to a portion in the vicinity of the end portion 20f of the belt 20 by a fixing portion 29. Note that the attached position of the fixing portion 29 with respect to the longitudinal direction X of the belt 20 (corresponding to the circumferential direction of the left wrist 90 in the worn state) is variable and is set in advance based on the circumferential length of the left wrist 90 of the subject 80. As a result, the sphygmomanometer 1 (belt 20) is formed to have a substantially annular shape as a whole, and the bottom surface 10b of the main body 10 and the end portion 20f of the belt 20 can be opened and closed by the buckle 24 in a direction indicated by an arrow B.

When wearing the sphygmomanometer 1 on the left wrist 90, the subject 80 inserts, in a direction indicated by an arrow A in FIG. 2, his or her left hand through the belt 20 in a large diameter annular state with the buckle 24 opened. Then, as illustrated in FIG. 3, the subject 80 adjusts the angular position of the belt 20 around the left wrist 90 to position the transmission and reception unit 40 of the belt 20 on the radial artery 91 running in the left wrist 90. As a result, the transmission and reception unit 40 (the transmission and reception antenna group 40E) faces a portion in the palm side surface 90a of the left wrist 90 corresponding to the radial artery 91. In this state, the subject 80 closes and fixes the buckle 24. In this way, the subject 80 can easily wear the sphygmomanometer 1 (belt 20) on the left wrist 90.

As illustrated in FIG. 3, in this example, the belt 20 includes a band body 20C forming the outer circumferential surface 20b, and the pressing cuff 21 attached along the inner circumferential surface 20a of the band body 20C. The band body 20C is made of a plastic material (in this example, a silicone resin having a thickness of 5 mm), and is flexible in a thickness direction Z but almost not elastic in the longitudinal direction X (corresponding to the circumferential direction of the left wrist 90) (substantially non-elastic) in this example. In this example, the pressing cuff21 is configured as a fluid bag obtained by facing two polyurethane sheets, which can be expanded and contracted, each other in the thickness direction Z, and welding circumferential edge portions of them. In this example, the pressing cuff 21 is attached to cover the first transmission antenna 41 and the first reception antenna 42, along the inner circumferential surface 20a of the band body 20C. Hereinafter, the band body 20C is referred to as a belt 20 unless otherwise specified.

In this example, as illustrated in FIG. 3, in the worn state, the transmission and reception antenna group 40E corresponds to the radial artery 91 in the circumferential direction of the left wrist 90. In particular, a pair of the first transmission antenna 41 and the first reception antenna 42 (hereinafter, referred to as a first transmission/reception antenna pair (41, 42)) faces the radial artery 91 with the pressing cuff21 provided in between. Furthermore, in this example, as illustrated in FIG. 5, when the blood pressure is measured (particularly, when the blood pressure is measured based on pulse wave transmit time described later), the subject 80 takes the following predetermined recommended measurement posture (denoted with the reference numeral PO). Specifically, the subject 80 raises a forearm 92 to diagonally cross the trunk 82 (with the hand up and elbow down), maintains the left wrist 90 to be at the same height level as the heart 81 with the palm side surface 90a of the left wrist 90 facing the heart 81 (thus, with the back side surface 90b of the left wrist 90 facing forward). As a result, as illustrated in FIG. 3, a pair of the second transmission antenna 43 and the second reception antenna 44 (hereinafter, referred to as a second transmission/reception antenna pair (43, 44)) face the heart 81.

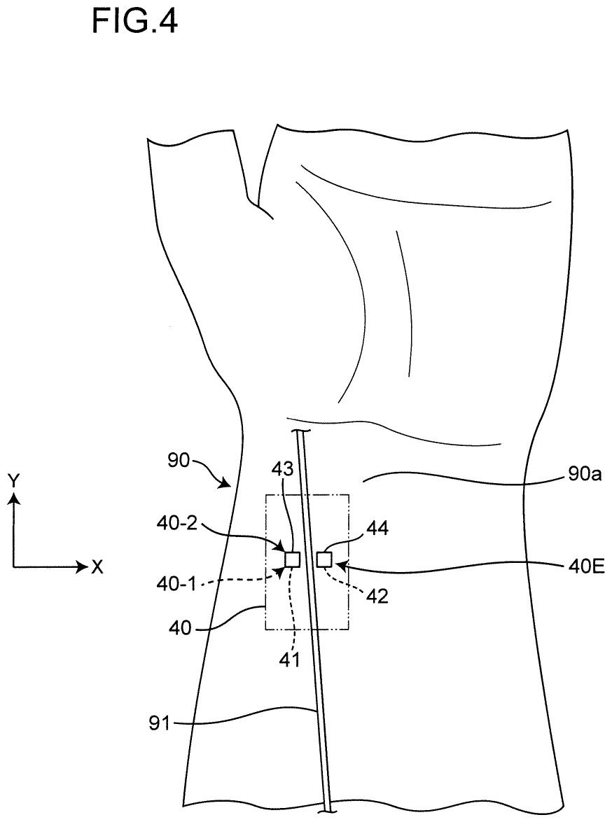

In this example, as illustrated in FIG. 4 (plan layout in the worn state), one transmission antenna or reception antenna has a 3×3 mm square shape in a planar direction (a direction along an XY plane in FIG. 4) so as to be capable of emitting and receiving radio waves at a frequency in a 24 GHz band (this shape in the planar direction will be referred to as a “pattern shape”). In this example, the distance between the center of the first transmission antenna 41 and the center of the first reception antenna 42 in the longitudinal direction X of the belt 20 is set to be in a range between 5 mm and 10 mm (8.5 mm in this example). Correspondingly, the distance between the center of the second transmission antenna 43 and the center of the second reception antenna 44 in the longitudinal direction X of the belt 20 is set to be in a range between 5 mm and 10 mm (8.5 mm in this example). The pattern shape of each of the transmission and reception antennas and the distance between the centers of the transmission/reception antennas are merely examples, and may be appropriately selected according to the size of the sphygmomanometer and the like.

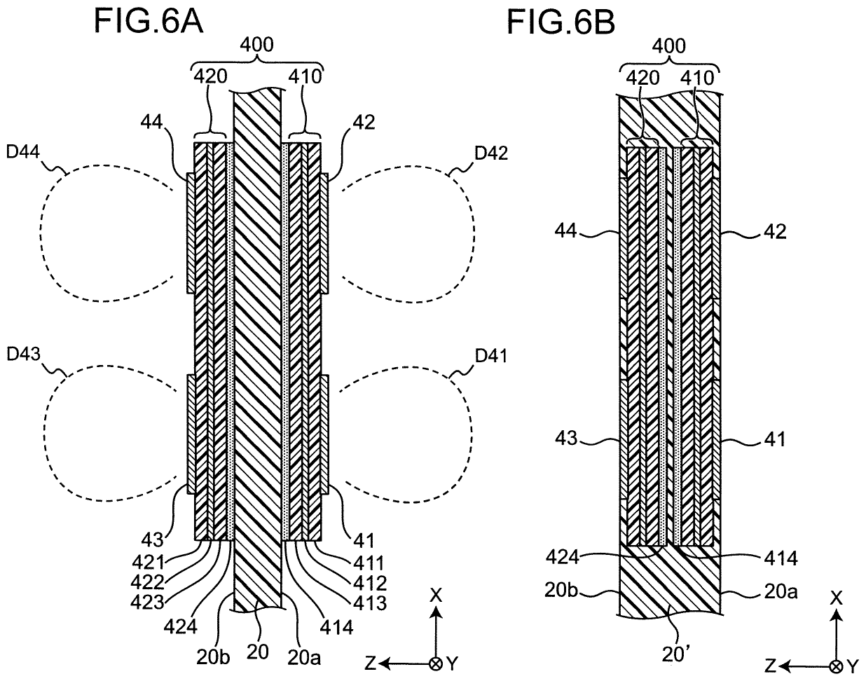

FIG. 6A illustrates a cross-sectional structure of the transmission and reception antenna group 40E. In this example, the first transmission/reception antenna pair (41, 42) and the second transmission/reception antenna pair (43, 44) are respectively attached to the inner circumferential surface 20a and the outer circumferential surface 20b of the belt 20 via substrates 410 and 420. In this example, the substrate 410 is formed with a copper layer 412 serving as a shielding layer having a thickness of 30 μm interposed between Flame Retardant Type 4 (FR4) layers 411 and 413 each having a thickness of 0.5 mm. On one surface (the surface on the −Z side) of the substrate 410, the transmission antenna 41 and the reception antenna 42 each made of a copper layer having a thickness of 30 μm are formed in a pattern. The opposite surface (the surface on the +Z side) of the substrate 410 is attached to the inner circumferential surface 20a of the belt 20 by an adhesive layer 414. Similarly, the substrate 420 is formed with a copper layer 422 serving as a shielding layer having a thickness of 30 μm interposed between FR4 layers 421 and 423 each having a thickness of 0.5 mm. On one surface (the surface on the +Z side) of the substrate 420, the transmission antenna 43 and the reception antenna 44 each made of a copper layer having a thickness of 30 μm are formed in a pattern. The opposite surface (the surface on the −Z side) of the substrate 420 is attached to the outer circumferential surface 20b of the belt 20 by an adhesive layer 424. In this structure, the directivity of the first transmission antenna 41 and the first reception antenna 42 spreads in the −Z direction as indicated by broken lines D41 and D42, respectively. On the other hand, the directivity of the second transmission antenna 43 and the second reception antenna 44 spreads in the +Z direction as indicated by broken lines D43 and D44, respectively. The copper layers 412 and 422 shield radio waves between the first transmission/reception antenna pair (41, 42) and the second transmission/reception antenna pair (43, 44). Thereby, interference between the first transmission/reception antenna pair (41, 42) and the second transmission/reception antenna pair (43, 44) is suppressed, whereby a pulse wave signal described later and a heartbeat signal can be acquired with high accuracy. Note that the substrates 410 and 420 constitute a base section 400 for the transmission and reception antenna group 40E. The shielding layer is not limited to a conductive material such as copper, but may have any layer shielding effect on radio waves.

As illustrated in FIG. 6B, a belt may be obtained by embedding each of the transmission antennas 41 and 43 and the reception antennas 42 and 44 in the belt 20 (denoted with a reference numeral 20′) so that the belt becomes flat on the inner circumferential surface 20a side and on the outer circumferential surface 20b side. In this example, the thickness of the belt 20′ is set to 8 mm. In this case, since the inner circumferential surface 20a side of the belt 20′ is flat, the subject 80 is free of uncomfortable feeling while wearing the belt 20′ (which may be felt if the belt has recesses and protrusions on the inner circumferential surface side). Furthermore, with the outer circumferential surface 20b side of the belt 20′ being flat, the transmission and reception antenna group 40E of the sphygmomanometer 1 is less likely to break even when the outer circumferential surface 20b of the belt 20′ comes into contact with a desk, a wall, or the like due to the activity of the subject 80. Furthermore, a better appearance can be achieved.

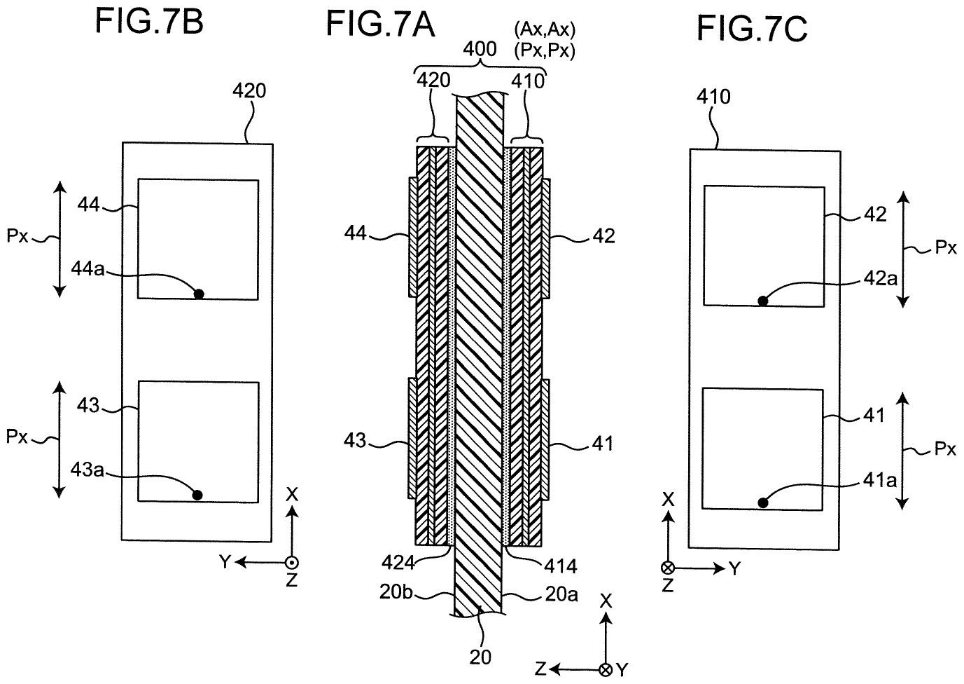

FIG. 7A illustrates a cross-sectional structure of the transmission and reception antenna group 40E corresponding to FIG. 6A. Note that (Ax, Ax) in the upper part of FIG. 7A represents an antenna arrangement in which the first transmission/reception antenna pair (41, 42) is arranged in the X direction, and the second transmission/reception antenna pair (43, 44) is arranged in the X direction. FIG. 7C illustrates the first transmission/reception antenna pair (41, 42) in FIG. 7A as viewed from the right side (−Z direction). In this example, in the first transmission antenna 41 and the first reception antenna 42, feed points 41a and 42a respectively connected to a transmission circuit and a reception circuit described later are provided at the center of the side on the −X side. Thus, linear polarization Px along the X direction is obtained as the polarization direction of the radio waves emitted from the first transmission antenna 41 and the polarization direction of the radio waves received by the first reception antenna 42, so that the transmission and reception between the antennas can be performed with a small amount of loss. FIG. 7B illustrates the second transmission/reception antenna pair (43, 44) in FIG. 7A as viewed from the left side (+Z direction). In this example, in the second transmission antenna 43 and the second reception antenna 44, feed points 43a and 44a respectively connected to a transmission circuit and a reception circuit described later are provided at the center of the side on the −X side. Thus, linear polarization Px is obtained as the polarization direction of the radio waves emitted from the second transmission antenna 43 and the polarization direction of the radio waves received by the second reception antenna 44, so that the transmission and reception between the antennas can be performed with a small amount of loss. Thus, a symbol (Px, Px) in the upper part of FIG. 7A represents such a combination with the polarization direction of the first transmission/reception antenna pair (41, 42) being the linear polarization Px, and the polarization direction of the second transmission/reception antenna pair (43, 44) being the linear polarization Px. The positions where each of the feed points 41a and 42a and the feed points 43a and 44a is arranged is not limited to the center of the corresponding side, and may be shifted from the center (the same applied to an example described later).

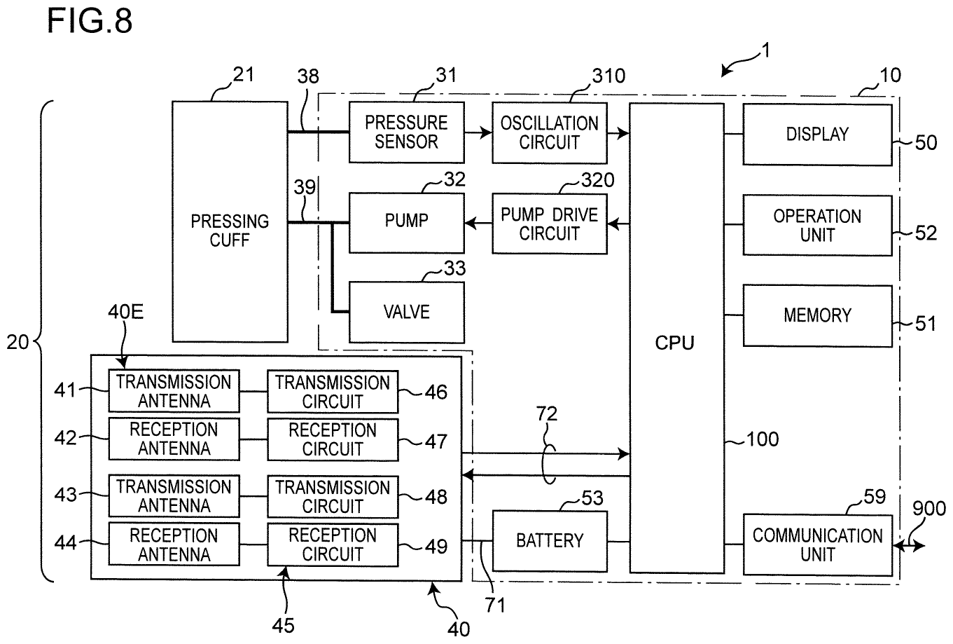

FIG. 8 illustrates an overall block configuration of a control system of the sphygmomanometer 1. The main body 10 of the sphygmomanometer 1 is provided with, in addition to the display 50 and the operation unit 52 described above, a Central Processing Unit (CPU) 100 serving as a control unit, a memory 51 serving as a storage unit, a communication unit 59, a pressure sensor 31, a pump 32, a valve 33, an oscillation circuit 310 that converts the output from the pressure sensor 31 into a frequency, and a pump drive circuit 320 that drives the pump 32. Further, the transmission and reception unit 40 includes a transmission and reception circuit group 45 controlled by the CPU 100 in addition to the transmission and reception antenna group 40E described above.

In this example, the display 50 is formed by an organic Electro Luminescence (EL) display, and displays information related to blood pressure measurement such as a blood pressure measurement result and other information, based on a control signal from the CPU 100. The display 50 is not limited to an organic EL display, and may be another type of display such as a Liquid Cristal Display (LCD).

In this example, the operation unit 52 is formed by a push-type switch, and inputs an operation signal corresponding to an instruction to start or stop blood pressure measurement by the subject 80, to the CPU 100. Note that the operation unit 52 is not limited to a push-type switch, and may be, for example, a touch panel switch of a pressure-sensitive (resistance) or proximity (capacitance) type. Furthermore, a microphone (not illustrated) may be provided so that an instruction to start the blood pressure measurement can be input by voice of the subject 80.

The memory 51 temporarily stores data of a program for controlling the sphygmomanometer 1, data used for controlling the sphygmomanometer 1, setting data for setting various functions of the sphygmomanometer 1, data of blood pressure value measurement results, and the like. The memory 51 is used as a work memory when the program is executed and the like.

The CPU 100 executes various functions as a control unit in accordance with the program for controlling the sphygmomanometer 1 stored in the memory 51. For example, when executing blood pressure measurement by the oscillometric method, the CPU 100 drives the pump 32 (and the valve 33) based on a signal from the pressure sensor 31 in response to an instruction to start the blood pressure measurement from the operation unit 52. In this example, the CPU 100 performs control to calculate a blood pressure value based on a signal from the pressure sensor 31.

The communication unit 59 is controlled by the CPU 100 to transmit predetermined information to an external device via a network 900, and to receive information from the external device via the network 900 and transfer it to the CPU 100. Communications via the network 900 may be wireless or wired communications. In this embodiment, the network 900 is the Internet. However, this should not be construed in a limiting sense, and may be another type of network such as an in-hospital Local Area Network (LAN) or may be one-to-one communication using a USB cable or the like. The communication unit 59 may include a micro USB connector.

The pump 32 and the valve 33 are connected to the pressing cuff 21 via an air pipe 39 and the pressure sensor 31 is connected to the pressing cuff21 via an air pipe 38. The air pipes 39 and 38 may be a single common pipe. The pressure sensor 31 detects the pressure in the pressing cuff 21 via the air pipe 38. In this example, the pump 32 is a piezoelectric pump, and supplies air as a pressurizing fluid to the pressing cuff 21 through the air pipe 39 in order to raise the pressure (cuff pressure) in the pressing cuff21. The valve 33 is mounted on the pump 32 and thus is controlled to be opened and closed in accordance with turning ON/OFF of the pump 32. Specifically, the valve 33 closes when the pump 32 is turned ON so that the air is contained inside the pressing cuff21, and opens when the pump 32 is turned OFF so that and the air in the pressing cuff21 is discharged to the atmosphere through the air pipe 39. The valve 33 has a check valve function, so that the discharged air does not backflow. The pump drive circuit 320 drives the pump 32 based on a control signal given from the CPU 100.

The pressure sensor 31 is a piezoresistive pressure sensor in this example, and detects the pressure of the belt 20 (pressing cuff 21) through the air pipe 38, with the atmospheric pressure serving as a reference (zero), and outputs the detection results as time series signals. The oscillation circuit 310 oscillates based on an electric signal value, corresponding to a change in electric resistance due to the piezoresistance effect from the pressure sensor 31, and outputs a frequency signal, having a frequency corresponding to the electric signal value of the pressure sensor 31, to the CPU 100. In this example, the output from the pressure sensor 31 is used for controlling the pressure of the pressing cuff21 and for calculating blood pressure values (including Systolic Blood Pressure (SBP) and Diastolic Blood Pressure (DBP)) based on an oscillometric method.

A battery 53 supplies power to elements in the main body 10. In this example, the elements include those of the CPU 100, the pressure sensor 31, the pump 32, the valve 33, the display 50, the memory 51, the communication unit 59, the oscillation circuit 310, and the pump drive circuit 320. The battery 53 also supplies power to the transmission and reception circuit group 45 of the transmission and reception unit 40 through wiring 71. The wiring 71, as well as wirings 72 for signals, is sandwiched between the band body 20C of the belt 20 and the pressing cuff 21, and extend between the main body 10 and the transmission and reception unit 40 along the longitudinal direction X of the belt 20.

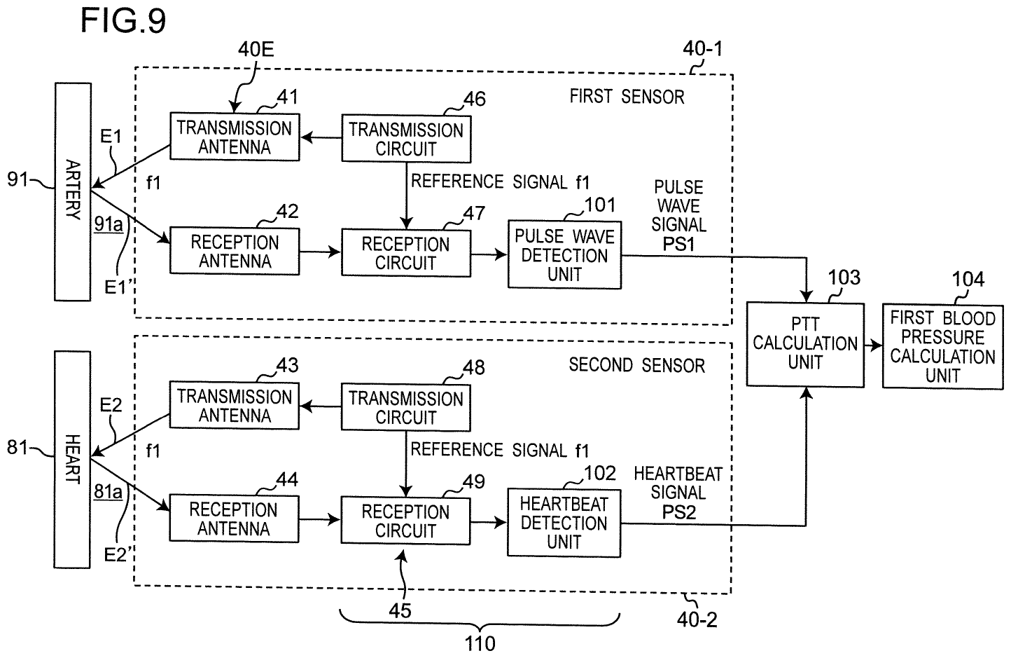

The transmission and reception circuit group 45 of the transmission and reception unit 40 includes transmission circuits 46 and 48 respectively connected to the transmission antennas 41 and 43, respectively, and reception circuits 47 and 49 respectively connected to the reception antennas 42 and 44. As illustrated in FIG. 9, the transmission circuit 46 under operation emits the radio waves E1 at a frequency f1 (f1=24.05 GHz in this example) in a 24 GHz band in this example, toward the radial artery 91 through the first transmission antenna 41 connected to the transmission circuit 46. The reception circuit 47 identifies the radio waves E1′ reflected by the radial artery 91 of the left wrist 90 and/or the tissue 91a being displaced in accordance with the pulse wave of the radial artery 91 based on a reference signal (frequency f1) from the transmission circuit 46, receives the waves via the first reception antenna 42, and detects and amplifies the waves. On the other hand, the transmission circuit 48 under operation emits the radio waves E2 at the same frequency f1 in the 24 GHz band in this example, toward the heart 81 via the second transmission antenna 43 connected with the circuit. The reception circuit 49 identifies the radio waves E2′ reflected by the heart 81 and/or the tissue 81a being displaced in accordance with the heartbeat of the heart 81 based on a reference signal (frequency f1) from the transmission circuit 48, receives the waves via the second reception antenna 44, and detects and amplifies the waves. In this example, with the transmission and reception circuit group 45 provided in the transmission and reception unit 40, a power feeding path from the transmission circuits 46 and 48 to the transmission antennas 41 and 43 can be made relatively short, where by degradation of the waveforms of the radio waves E1 and E2 can be suppressed. Furthermore, a reception path from the respective reception antennas 42 and 44 to the reception circuits 47 and 49 can be made relatively short. As a result, a pulse wave signal and a heartbeat signal described later can be acquired with high accuracy. In the following description, for the sake of simplicity, the reflected radio waves E1′ are assumed to be radio waves reflected by the radial artery 91, and the reflected radio waves E2′ are assumed to be radio waves reflected by the heart 81.

As will be described later in detail, a pulse wave detection unit 101 illustrated in FIG. 9 acquires the pulse wave signal PS1 representing the pulse wave of the radial artery 91 running in the left wrist 90 based on the output from the reception circuit 47. A heartbeat detection unit 102 acquires the heartbeat signal PS2 representing the heartbeat of the heart 81 based on the output from the reception circuit 49. Further, a pulse transit time (PTT) calculation unit 103 serving as a time difference acquisition unit calculates PTT as a time difference between the pulse wave signal PS1 and the heartbeat signal PS2 acquired by the pulse wave detection unit 101 and the heartbeat detection unit 102, respectively. A first blood pressure calculation unit 104 calculates a blood pressure value based on the pulse transit time acquired by the PTT calculation unit 103 using a predetermined correspondence formula between the pulse transit time and the blood pressure. Here, the pulse wave detection unit 101, the heartbeat detection unit 102, the PTT calculation unit 103, and the first blood pressure calculation unit 104 are implemented by the CPU 100 executing a predetermined program stored in the memory 51, for example. The first transmission antenna 41, the first reception antenna 42, the transmission circuit 46, the reception circuit 47, and the pulse wave detection unit 101 will be referred to as a first sensor 40-1. The second transmission antenna 43, the second reception antenna 44, the transmission circuit 48, the reception circuit 49, and the heartbeat detection unit 102 will be referred to as a second sensor 40-2. The transmission and reception circuit group 45, the pulse wave detection unit 101, and the heartbeat detection unit 102 correspond to the above-described vital sign detection unit 110.

In operation, the pulse wave detection unit 101 of the first sensor 40-1 and the heartbeat detection unit 102 of the second sensor 40-2 respectively output in time series, the pulse wave signal PS1 and the heartbeat signal PS2 with a mountain-like waveform as illustrated in FIG. 13, based on the outputs from the reception circuits 47 and 49. The heartbeat signal PS2 indicates a change in distance between the second transmission/reception antenna pair (43, 44) and the heart 81 due to the heartbeat. The pulse wave signal PS1 indicates a change in distance between the first transmission/reception antenna pair (41, 42) and the radial artery 91 due to a pulse wave (resulting in expansion and contraction of a blood vessel). The heartbeat signal PS2 appears earlier than the pulse wave signal PS1.

In this example, in operation with the recommended measurement posture PO taken as illustrated in FIG. 5, the distance between the first transmission/reception antenna pair (41, 42) and the radial artery 91 is expected to be about 5 mm, and the distance between the second transmission/reception antenna pair (43, 44) and the heart 81 is assumed to be about 50 mm. Based on these distances, the intensity levels of the radio waves emitted by the first transmission antenna 41 and the second transmission antenna 43 are about 0.5 mW and about 10 mW, respectively. The reception levels of the reception antennas 42 and 44 are about 1 μW (−30 dBm in decibel value with respect to 1 mW) and about 0.2 μW, respectively. The output level of each of the reception circuits 47 and 49 is about 1 volt. Furthermore, the intensity levels at peaks A1 and A2 of the pulse wave signal PS1 and the heartbeat signal PS2 are each about 100 mV to 1 volt. With such a setting, the pulse wave signal PS1 and the heartbeat signal PS2 can be acquired with high accuracy.

For example, under a condition that the distance along the artery from the heart 81 to the left wrist 90 is 70 cm and the Pulse Wave Velocity (PWV) is in a range of 1000 cm/s to 2000 cm/s, a time difference Δt between the heartbeat signal PS2 and the pulse wave signal PS1 is in a range between 35 ms and 70 ms.

### (Configuration and Operation for Blood Pressure Measurement by Oscillometric Method)

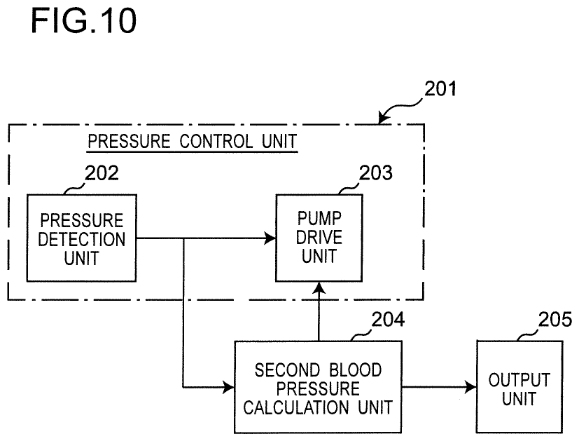

FIG. 10 illustrates a block configuration implemented in the sphygmomanometer 1 by a program for performing the oscillometric method.

In this block configuration, a pressure control unit 201, a second blood pressure calculation unit 204, and an output unit 205 are mainly implemented.

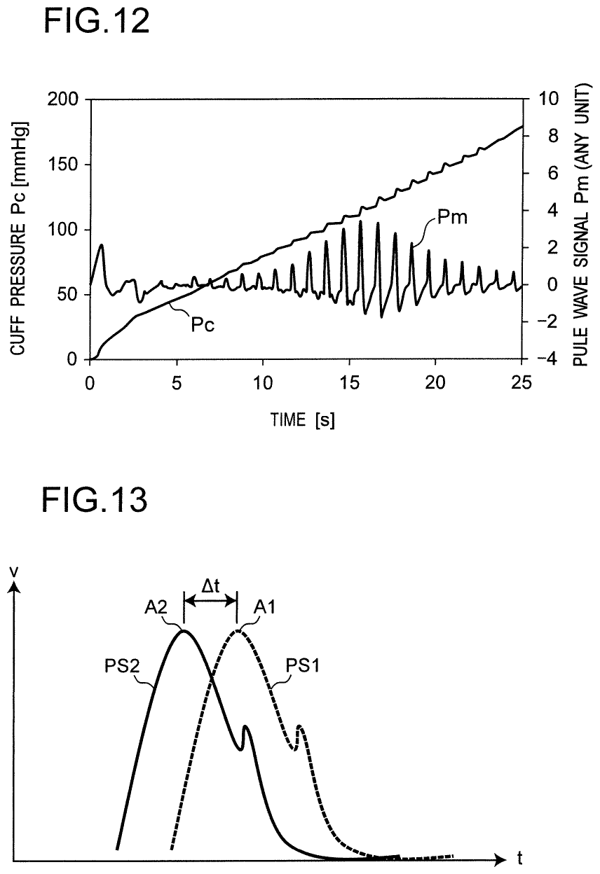

Furthermore, the pressure control unit 201 includes a pressure detection unit 202 and a pump drive unit 203. The pressure detection unit 202 processes the frequency signal input from the pressure sensor 31 through the oscillation circuit 310, and performs a process for detecting the pressure (cuff pressure) in the pressing cuff 21. The pump drive unit 203 performs a process for driving the pump 32 and the valve 33 through the pump drive circuit 320 based on cuff pressure Pc detected (see FIG. 12). Thus, the pressure control unit 201 controls the pressure by supplying air to the pressing cuff 21 at a predetermined pressurization speed.

The second blood pressure calculation unit 204 performs a process including: acquiring a variation component of the arterial volume included in the cuff pressure Pc as a pulse wave signal Pm (see FIG. 12); and based on the acquired pulse wave signal Pm, calculating blood pressure values (the systolic blood pressure SBP and diastolic blood pressure DBP) through the oscillometric method with a known algorithm applied. When the calculation of the blood pressure value is completed, the second blood pressure calculation unit 204 causes the pump drive unit 203 to stop.

The output unit 205 performs a process of displaying the calculated blood pressure values (systolic blood pressure SBP and diastolic blood pressure DBP) on the display 50 in this example.

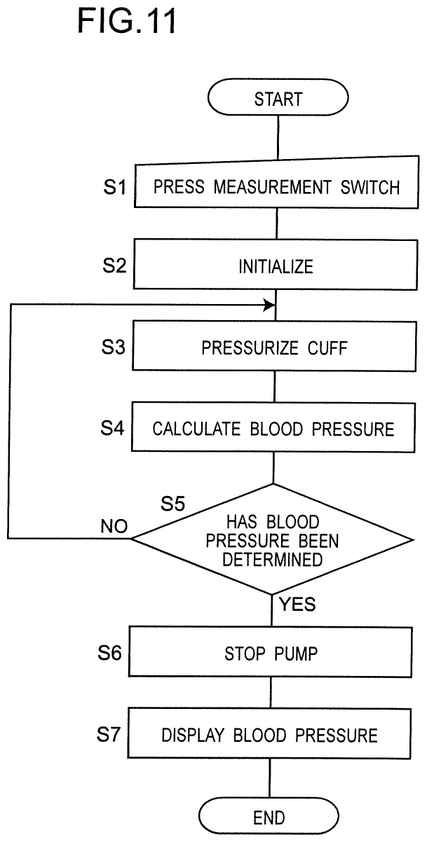

FIG. 11 illustrates an operation flow (flow of the blood pressure measurement method) when the sphygmomanometer 1 measures the blood pressure through the oscillometric method. The belt 20 of the sphygmomanometer 1 is assumed to be worn around the left wrist 90 of the subject 80 in advance. The subject 80 is assumed to be taking the recommended measurement posture PO illustrated in FIG. 5.

When the subject 80 instructs the blood pressure measurement through the oscillometric method by using the push-type switch as the operation unit 52 provided on the main body 10 (step S1 in FIG. 11), the CPU 100 starts the operation and initializes the processing memory area (step S2). Furthermore, the CPU 100 uses the pump drive circuit 320 to turn off the pump 32 and open the valve 33, to discharge the air in the pressing cuff21. Next, control is performed to set the present output value of the pressure sensor 31 as a value corresponding to the atmospheric pressure (0 mmHg adjustment).

Next, the CPU 100 performs control to send air to the pressing cuff 21, by functioning as the pump drive unit 203 of the pressure control unit 201 to close the valve 33, and then using the pump drive circuit 320 to drive the pump 32. As a result, the pressing cuff21 is inflated with the cuff pressure Pc (see FIG. 12) gradually increasing, so that the left wrist 90 as the measurement target part is pressurized (step S3 in FIG. 11).

In this pressurization process, the CPU 100 functions as the pressure detection unit 202 of the pressure control unit 201 to calculate the blood pressure value, uses the pressure sensor 31 to monitor the cuff pressure Pc, and acquires the artery volume variation component produced in the radial artery 91 in the left wrist 90 as the pulse wave signal Pm as illustrated in FIG. 12.

Next, in step S4 in FIG. 11, the CPU 100 functions as the second blood pressure calculation unit and attempts to calculate the blood pressure values (the systolic blood pressure SBP and diastolic blood pressure DBP) through the oscillometric method with a known algorithm applied, based on currently acquired the pulse wave signal Pm.

At this point, when the blood pressure value cannot be calculated yet due to lack of data (NO in step S5), the processes in step S3 to S5 are repeated as long as the cuff pressure Pc has not reached the upper limit pressure (determined in advance to be 300 mmHg for example, for the sake of safety).

When the blood pressure value is successfully calculated (YES in step S5), the CPU 100 performs control to stop the pump 32 and open the valve 33 to discharge the air in the pressing cuff 21 (step S6). Finally, the CPU 100 functions as the output unit 205 and displays the measurement result of the blood pressure value on the display 50 and records the result in the memory 51 (step S7).

The calculation of the blood pressure value is not limited to the pressurization process, and may be performed in the depressurization process.

### (Blood Pressure Measurement Based on Pulse Transit Time)

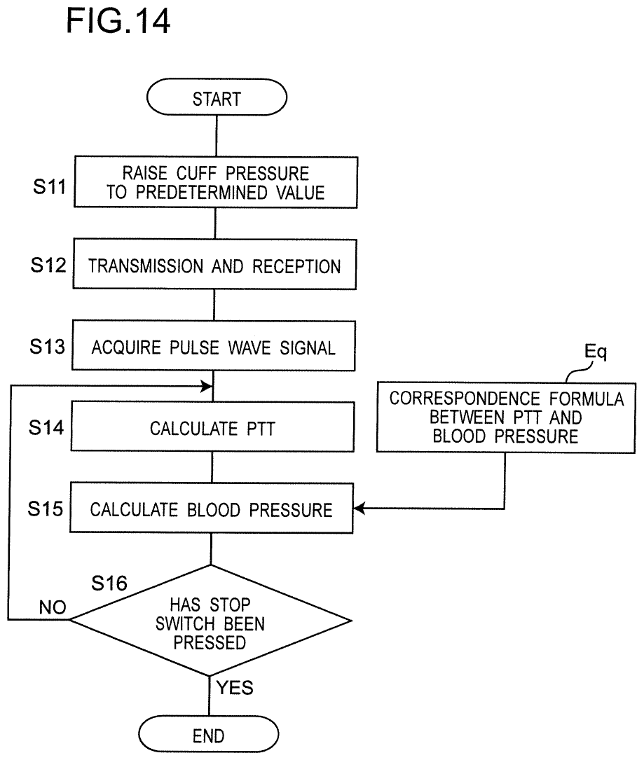

FIG. 14 illustrates an operation flow according to the vital sign measurement method and the blood pressure measurement method according to one embodiment of the present invention. In the flow, the sphygmomanometer 1 acquires PTT and the blood pressure measurement (estimation) is performed based on the pulse transit time. The belt 20 of the sphygmomanometer 1 is assumed to be worn around the left wrist 90 of the subject 80 in advance. The subject 80 is assumed to be taking the recommended measurement posture PO illustrated in FIG. 5.

When the subject 80 instructs the blood pressure measurement based on the PTT by using the push-type switch as the operation unit 52 provided on the main body 10, the CPU 100 starts the operation. Specifically, as illustrated in step S11 of FIG. 14, the CPU 100 performs the control to send air to the pressing cuff21 by closing the valve 33 and using the pump drive circuit 320 to drive the pump 32 via the pump drive circuit 320, to inflate the pressing cuff 21 while raising the cuff pressure Pc to a predetermined value. In this example, in order to reduce the physical burden on the subject 80, the pressurization is limited to a pressure (for example, about 5 mmHg) that is sufficient for the belt 20 to be in close contact with the left wrist 90. Thus, the pressing cuff21 is reliably brought into contact with the palm side surface 90a of the left wrist 90, so that the distance between the first transmission/reception antenna pair (41, 42) and the radial artery 91 is prevented from varying up and down due to the body movement of the subject 80. Note that step S11 may be omitted.

Next, in this worn state, as illustrated in step S12 in FIG. 14, the CPU 100 controls transmission and reception respectively in the first sensor 40-1 and the second sensor 40-2 illustrated in FIG. 9. Specifically, in the first sensor 40-1, the transmission circuit 46 emits the radio waves E1 to the radial artery 91 through the first transmission antenna 41. At the same time, the reception circuit 47 identifies the radio waves E1′ reflected by the radial artery 91 based on the reference signal (frequency f1) from the transmission circuit 46, receives the waves via the first reception antenna 42, and detects and amplifies the waves. In the second sensor 40-2, the transmission circuit 48 emits the radio waves E2 to the heart 81 through the second transmission antenna 43. At the same time, the reception circuit 49 identifies the radio waves E2′ reflected by the heart 81 based on the reference signal (frequency f1) from the transmission circuit 48, receives the waves via the second reception antenna 44, and detects and amplifies the waves.

Next, as illustrated in step S13 in FIG. 14, the CPU 100 functions as the pulse wave detection unit 101 and the heartbeat detection unit 102 respectively for the first sensor 40-1 and the second sensor 40-2 illustrated in FIG. 9, to acquire the pulse wave signal PS1 and the heartbeat signal PS2 as illustrated in FIG. 13. Specifically, for the first sensor 40-1, the CPU 100 functions as the pulse wave detection unit 101, and acquires the pulse wave signal PS1 representing the pulse wave of the radial artery 91 from the output from the reception circuit 47 through the systolic and diastolic periods of the artery. Furthermore, for the second sensor 40-2, the CPU 100 functions as the heartbeat detection unit 102, and acquires the heartbeat signal PS2 representing the heartbeat of the heart 81 from the output from the reception circuit 49 through the systolic and diastolic periods of the heart.

Next, as illustrated in step S14 of FIG. 14, the CPU 100 functions as the PTT calculation unit 103 serving as the time difference acquisition unit, and calculates the time difference between the heartbeat signal PS2 and the pulse wave signal PS1 as the PIT. More specifically, in this example, a time difference Δt between the peak A2 of the heartbeat signal PS2 and the peak A1 of the pulse wave signal PS1 illustrated in FIG. 13 is acquired as the PT.

Thereafter, as illustrated in step S15 in FIG. 14, the CPU 100 functions as the first blood pressure calculation unit, calculates (estimates) the blood pressure based on the PTT acquired in step S14, by using a predetermined correspondence formula Eq between the PTT and the blood pressure. Here, this predetermined correspondence formula Eq between the PTT and the blood pressure is provided as a known fraction function including an item 1/DT² as in

EBP=α/DT²+β (Eq.1),

where DT represents PTT and EBP represents the blood pressure (and α and β each represents a known coefficient or constant) (see, for example, JP-A-10-201724). Furthermore, as the predetermined correspondence formula Eq between the PTT and the blood pressure, another known correspondence formula such as a formula including an item 1/DT and an item DT in addition to the item 1/DT² can be used. That is,

EBP=α/DT²+β/DT+γDT+δ (Eq.2)

(α, β, γ, and δ each represents a known coefficient or constant).

When the blood pressure is calculated (estimated) in this manner, the pulse wave signal PS1 representing a pulse wave of the radial artery 91 in the left wrist 90 and the heartbeat signal PS2 representing the heartbeat of the heart 81 are acquired and the blood pressure value is calculated, with a simple physical condition in which the subject 80 wears the belt 20 around the left wrist 90 and takes the predetermined recommended measurement posture PO. In other words, measurement can be performed without mounting or attaching electrodes to portions sandwiching the heart 81 of the subject 80. The subject 80 can easily wear the sphygmomanometer 1 (belt 20) on the left wrist 90 simply by inserting the left wrist 90 through the belt 20 and closing the buckle 24. Furthermore, the recommended measurement posture PO taken by the subject 80 may include a wide variety of postures such as a posture with the upper body erected or a lying posture, and thus a degree of freedom is high. Therefore, the sphygmomanometer 1 imposes a small physical burden on the subject 80 for the measurement. The measurement result of the blood pressure value is displayed on the display 50 and is recorded in the memory 51.

In this example, as long as an instruction to stop the measurement is not issued using the push-type switch as the operation unit 52 in step S16 of FIG. 14 (NO in step S16), the calculation of the PTT (step S14 in FIG. 14) and the calculation (estimation) of the blood pressure (step S15 in FIG. 14) are periodically repeated at each time the pulse wave signal PS1 and the heartbeat signal PS2 are input in accordance with the pulse wave and the heartbeat. The CPU 100 updates the measurement result of the blood pressure value, and displays it on the display 50, and stores and records it in the memory 51. Then, when the instruction to stop the measurement is issued in step S16 of FIG. 14 (YES in step S16), the measurement operation ends.

With the sphygmomanometer 1, by measuring the blood pressure based on the PTT, the blood pressure can be continuously measured over a long period of time while imposing only a small physical burden on the subject 80.

Furthermore, in the sphygmomanometer 1, the transmission and reception unit 40 and the main body 10 (including the CPU 100 and the like) are provided integrally with the belt 20. Thus, the blood pressure measurement (estimation) based on the PTT and the blood pressure measurement through the oscillometric method can be performed by an integrated device using the common belt 20. Therefore, usability for the subject 80 as the user can be improved. For example, generally, when blood pressure measurement (estimation) based on PTT is performed, the correspondence formula Eq between the PTT and blood pressure needs to be calibrated as appropriate (in the above example, values such the coefficients α and β based on the PTT and the blood pressure value are updated). In this context, with this sphygmomanometer 1, the blood pressure is measured by the oscillometric method using the same apparatus, and the calibration of the correspondence formula Eq can be performed based on the result of the measurement, so that usability for the subject 80 can be improved. In addition, the PTT method (blood pressure measurement based on PTT) enabling continuous measurement but with low accuracy may be performed to capture sharp blood pressure rise, and using the sharp blood pressure rise as a trigger, more accurate measurement through the oscillometric method can be started.

In particular, with the sphygmomanometer 1, no wiring needs to be extended to the outside of the belt 20 to obtain the pulse wave signal PS1, the heartbeat signal PS2, the PTT, and the blood pressure value from the outputs of the reception antennas 42 and 44. Thus, with the sphygmomanometer 1, the subject 80 needs not be bothered by the wiring cable at the time of the measurement, and thus the physical load is small.

In the above example, the first transmission antenna 41 and the first reception antenna 42 are provided separately from each other, but the present invention is not limited to this. An antenna element, which is a simple substance in terms of space, may be used as a transmission antenna and a reception antenna (that is, an antenna used for both transmission and reception) via a known circulator. The same applies to the second transmission antenna 43 and the second reception antenna 44.

### (Modification 1; Variation in Frequency)

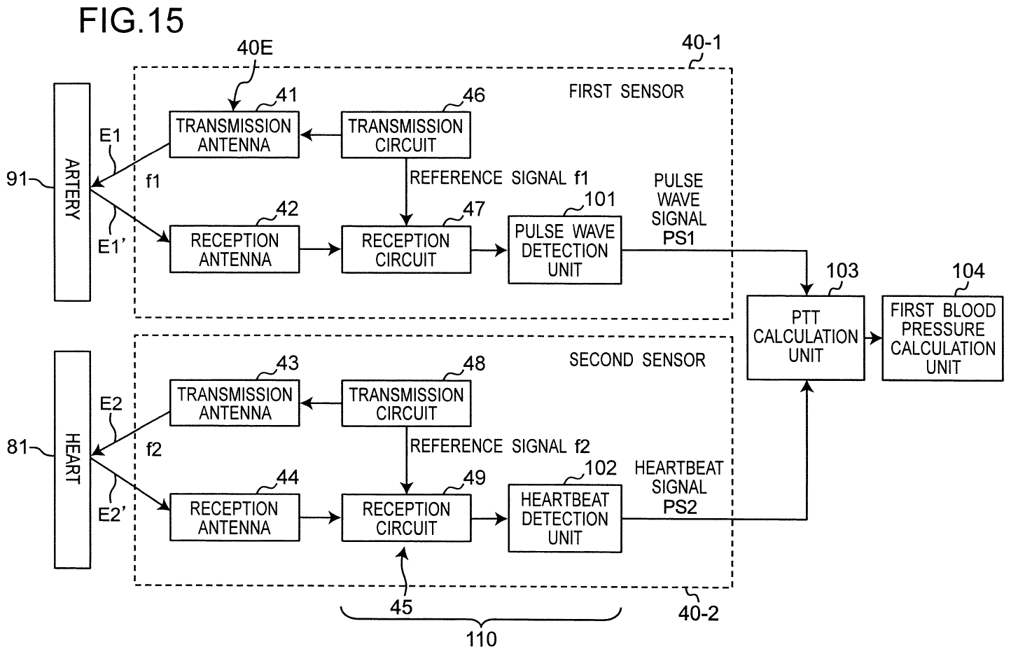

In the above example, as illustrated in FIG. 9, the frequency of the radio waves E1 emitted from the transmission antenna 41 toward the radial artery 91 of the left wrist 90 and the frequency of the radio waves E2 emitted from the transmission antenna 43 toward the heart 81 are assumed to be the same frequency f1. However, the present invention is not limited to this. For example, as illustrated in FIG. 15, the frequency of the radio waves E1 emitted from the transmission antenna 41 toward the radial artery 91 of the left wrist 90 and the frequency of the radio waves E2 emitted from the transmission antenna 43 toward the heart 81 may respectively be the frequency f1 and a frequency f2 that are different from each other. In this example, f1=24.05 GHz and f2=24.25 GHz.

In the example of FIG. 15, the transmission circuit 46 under operation emits the radio waves E1 at the frequency f1 (=24.05 GHz) to the radial artery 91 via the first transmission antenna 41 connected to the transmission circuit 46. At the same time, the reception circuit 47 identifies the radio waves E1′ reflected by the radial artery 91 of the left wrist 90 based on the reference signal (frequency f1) from the transmission circuit 46, receives the waves via the first reception antenna 42, and detects and amplifies the waves. On the other hand, the transmission circuit 48 under operation emits the radio waves E2 at the frequency f2 (=24.25 GHz) to the heart 81 via the second transmission antenna 43 connected to the transmission circuit 48. At the same time, the reception circuit 49 identifies the radio waves E2′ reflected by the heart 81 based on the reference signal (frequency f2) from the transmission circuit 48, receives the waves via the second reception antenna 44, and detects and amplifies the waves.

In this case, the radio waves E1′ reflected by the radial artery 91 of the left wrist 90 and the radio waves E2′ reflected by the heart 81 can be distinguishable from each other based on the frequencies f1 and f2 to be prevented from interfering. As a result, the pulse wave signal PS1 and the heartbeat signal PS2 can be acquired with high accuracy.

### (Modification 2; Variation in Antenna Arrangement)

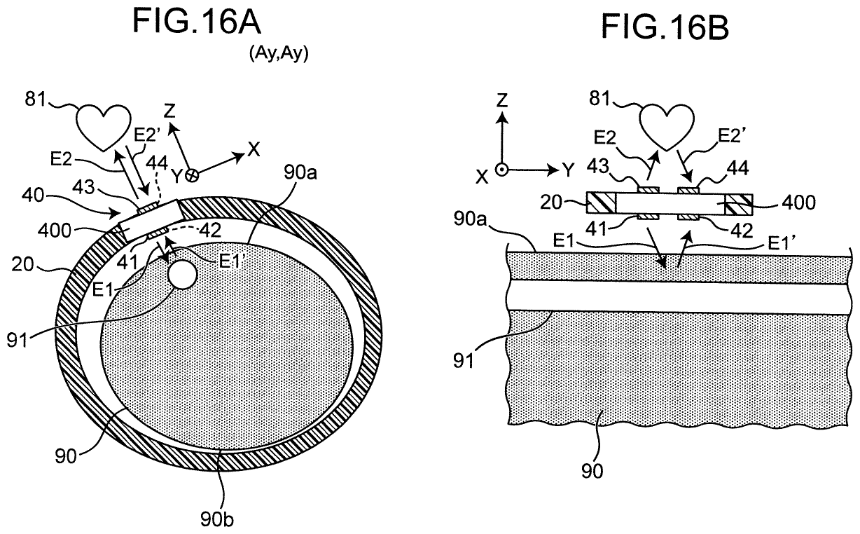

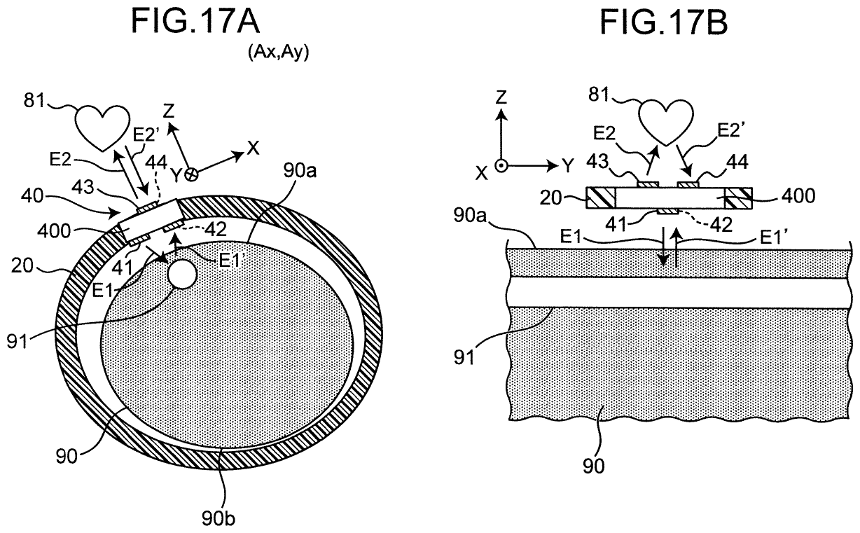

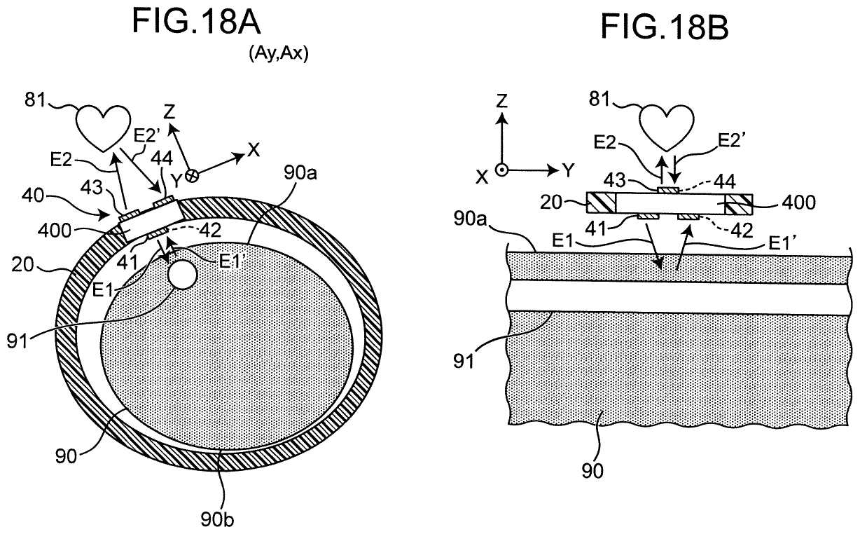

In the above example, the antenna arrangement (Ax, Ax) (the sign of this antenna arrangement is provided in the upper portion in FIG. 7A) is employed in which the first transmission/reception antenna pair (41, 42) are arranged side by side in the X direction and the second transmission/reception antenna pair (43, 44) are arranged side by side in the X direction as illustrated in FIG. 3 for example. However, the present invention is not limited to this. In the above example, an antenna arrangement (Ay, Ay) (the sign of this antenna arrangement is provided in the upper portion in FIG. 16A) may be employed in which the first transmission/reception antenna pair (41, 42) are arranged side by side in the Y direction and the second transmission/reception antenna pair (43, 44) are arranged side by side in the Y direction, as illustrated in FIGS. 16A and 16B. Here, FIG. 16A illustrates a cross section (ZX plane) orthogonal to the longitudinal direction of the left wrist 90 corresponding to FIG. 3. FIG. 16B illustrates a cross section (YZ plane) of what is illustrated in FIG. 16A (the same applies to FIG. 17 and FIG. 18 described below) taken along the longitudinal direction of the left wrist 90. Also, for the sake of simplicity, the main body 10 and the pressing cuff 21 is omitted in the figure (the same applies hereinafter).

Furthermore, an antenna arrangement (Ax, Ay) (the sign of this antenna arrangement is provided in the upper portion in FIG. 17A) is employed in which the first transmission/reception antenna pair (41, 42) are arranged side by side in the X direction and the second transmission/reception antenna pair (43, 44) are arranged side by side in the Y direction, as illustrated in FIGS. 17A and 17B.

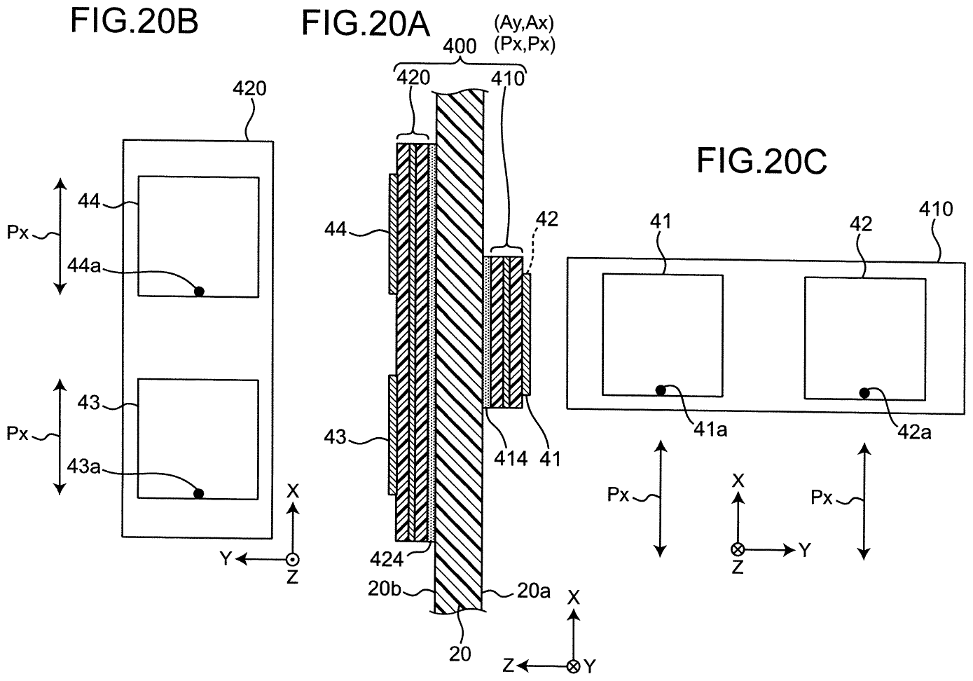

Furthermore, an antenna arrangement (Ay, Ax) (the sign of this antenna arrangement is provided in the upper portion in FIG. 18A) is employed in which the first transmission/reception antenna pair (41, 42) are arranged side by side in the Y direction and the second transmission/reception antenna pair (43, 44) are arranged side by side in the X direction, as illustrated in FIGS. 18A and 18B. In this example, as illustrated in FIG. 20B and FIG. 20C, the polarization direction of the first transmission/reception antenna pair (41, 42) are assumed to be the linear polarization Px, and the polarization direction of the second transmission/reception antenna pair (43, 44) are assumed to be the linear polarization Px. In the upper portion of FIG. 20A, the combination of the polarization directions is represented by a sign (Px, Px), together with the sign (Ay, Ax) of the antenna arrangement.

Also by employing these antenna arrangements (Ay, Ay), (Ax, Ay), and (Ay, Ax), the radio waves E1 can be emitted from the first transmission antenna 41 to the radial artery 91, and the radio waves E1′ reflected by the radial artery 91 can be received through the first reception antenna 42. Furthermore, the radio waves E2 can be emitted from the second transmission antenna 43 toward the heart 81, and the radio waves E2′ reflected by the heart 81 can be received through the reception antenna 44. With such a setting, the pulse wave signal PSI1 and the heartbeat signal PS2 can be acquired with high accuracy.

### (Modification 3; Variation in Polarization Direction)

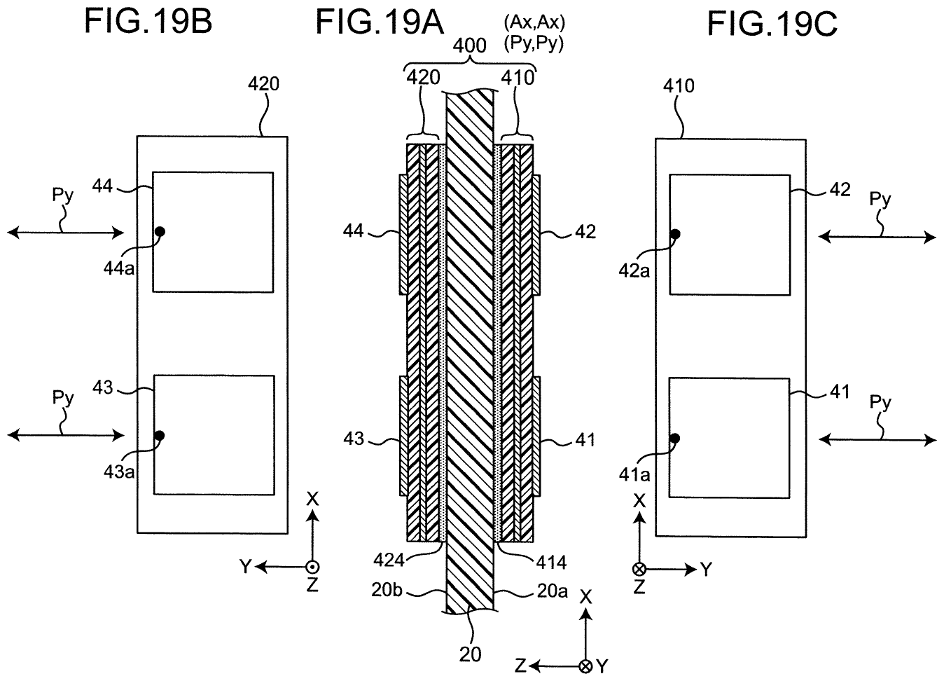

In the example described above, the polarization direction of the first transmission/reception antenna pair (41, 42) is the linear polarization Px, and the polarization direction of the second transmission/reception antenna pair (43, 44) is the linear polarization Px as illustrated in FIG. 7C and FIG. 7B for example (the symbol (Px, Px) in the upper portion of FIG. 7A represents the combination of such polarization directions). However, the present invention is not limited to this. For example, as illustrated in FIG. 19C, in the first transmission antenna 41 and the first reception antenna 42, the feed points 41a and 42a may be respectively provided at the centers of the sides on the −Y side, so that linear polarization Py is obtained as the polarization direction of the first transmission/reception antenna pair (41, 42). Similarly, as illustrated in FIG. 19B, in the second transmission antenna 43 and the second reception antenna 44, the feed points 43a and 44a may be respectively provided at the centers of the sides on the +Y side, so that the linear polarization Py is obtained as the polarization direction of the second transmission/reception antenna pair (43, 44). Thus, the transmission and reception between the first transmission antenna 41 and the first reception antenna 42 as well as the transmission and reception between the second transmission antenna 43 and the second reception antenna 44 can be performed with low loss. As a result, the pulse wave signal PS1 and the heartbeat signal PS2 can be acquired with high accuracy. The sign (Py, Py) in the upper portion of FIG. 19A indicates the combination of such polarization directions.

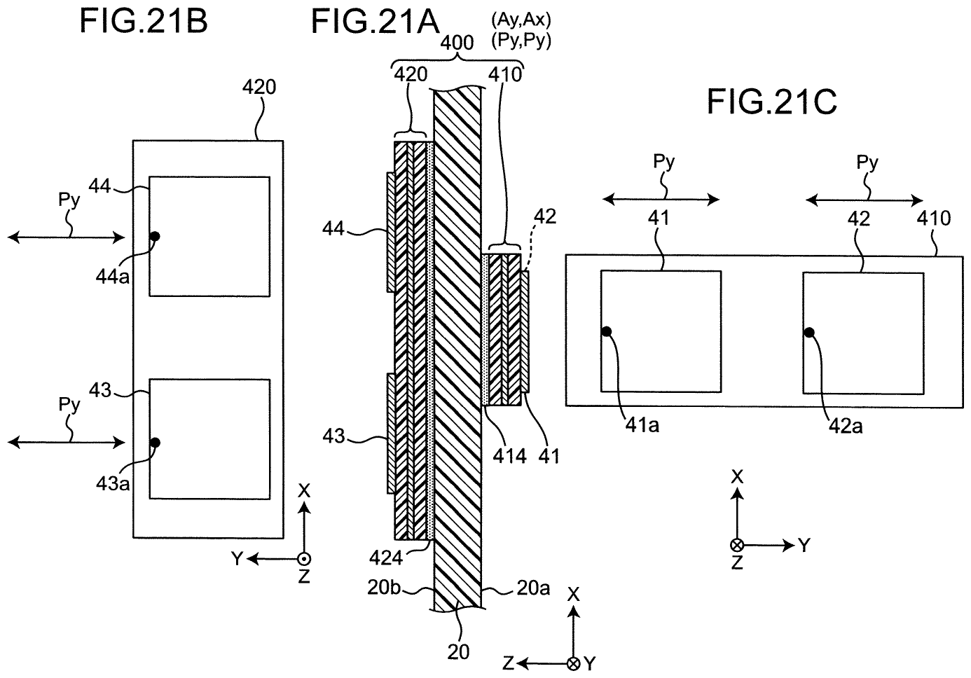

For the same reason as in the above example, for example, when the antenna arrangement (Ay, Ax) in which the first transmission/reception antenna pair (41, 42) are arranged side by side in the Y direction and the second transmission/reception antenna pair (43, 44) are arranged side by side in the X direction is employed as illustrated in FIG. 21A, the polarization direction of the first transmission/reception antenna pair (41, 42) may be the linear polarization Py, and the polarization direction of the second transmission/reception antenna pair (43, 44) may be the linear polarization Py as illustrated in FIGS. 21C and 21B for example. In the upper portion of FIG. 21A, the combination of the polarization directions is represented by a sign (Py, Py), together with the sign (Ay, Ax) of the antenna arrangement.

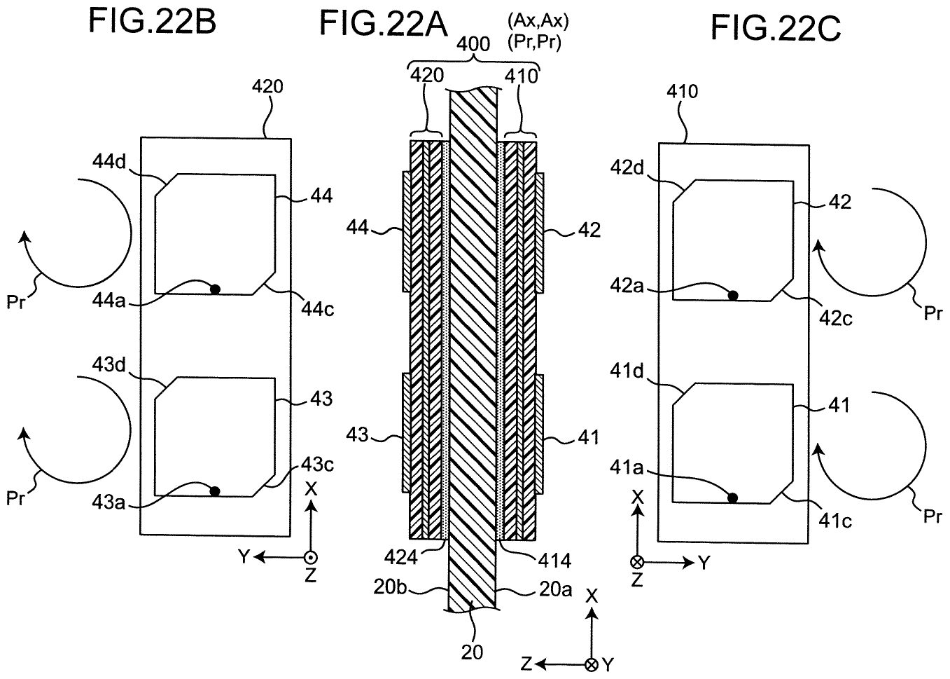

For the same reason as in the above example, for example, when the antenna arrangement (Ax, Ax) in which the first transmission/reception antenna pair (41, 42) are arranged side by side in the X direction and the second transmission/reception antenna pair (43, 44) are arranged side by side in the X direction is employed as illustrated in FIG. 22A, in the first transmission antenna 41, the feed point 41a may be provided at the center of the side on the −X side, a notch (perturbation element) 41c may be provided at the corner formed by the side on the −X side and the side on the +Y side, and a notch (perturbation element) 41d may be provided at the corner formed by the side on the +X side and the side on the −Y side as illustrated in FIG. 22C, so that clockwise circular polarization Pr can be obtained as the polarization direction of the first transmission antenna 41. Furthermore, in the first reception antenna 42, the feed point 42a may be provided at the center of the side on the −X side, a notch (perturbation element) 42c may be provided at the corner formed by the side on the −X side and the side on the +Y side, and a notch (perturbation element) 42d may be provided at the corner formed by the side on the +X side and the side on the −Y side, so that the clockwise circular polarization Pr can be obtained as the polarization direction of the first reception antenna 42. In this manner, the clockwise circular polarization Pr may be obtained as the polarization direction of the first transmission/reception antenna pair (41, 42). Similarly, in the second transmission antenna 43, the feed point 43a may be provided at the center of the side on the −X side, a notch (perturbation element) 43c may be provided at the corner formed by the side on the −X side and the side on the −Y side, and a notch (perturbation element) 43d may be provided at the corner formed by the side on the +X side and the side on the +Y side as illustrated in FIG. 22B, so that the clockwise circular polarization Pr can be obtained as the polarization direction of the second reception antenna 43. Furthermore, in the second reception antenna 44, the feed point 44a may be provided at the center of the side on the −X side, a notch (perturbation element) 44c may be provided at the corner formed by the side on the −X side and the side on the −Y side, and a notch (perturbation element) 44d may be provided at the corner formed by the side on the +X side and the side on the +Y side, so that the clockwise circular polarization Pr can be obtained as the polarization direction of the second reception antenna 44. In this manner, the clockwise circular polarization Pr may be obtained as the polarization direction of the second transmission/reception antenna pair (43, 44). In the upper portion of FIG. 22A, the combination of the polarization directions is represented by a sign (Pr, Pr), together with the sign (Ax, Ax) of the antenna arrangement.

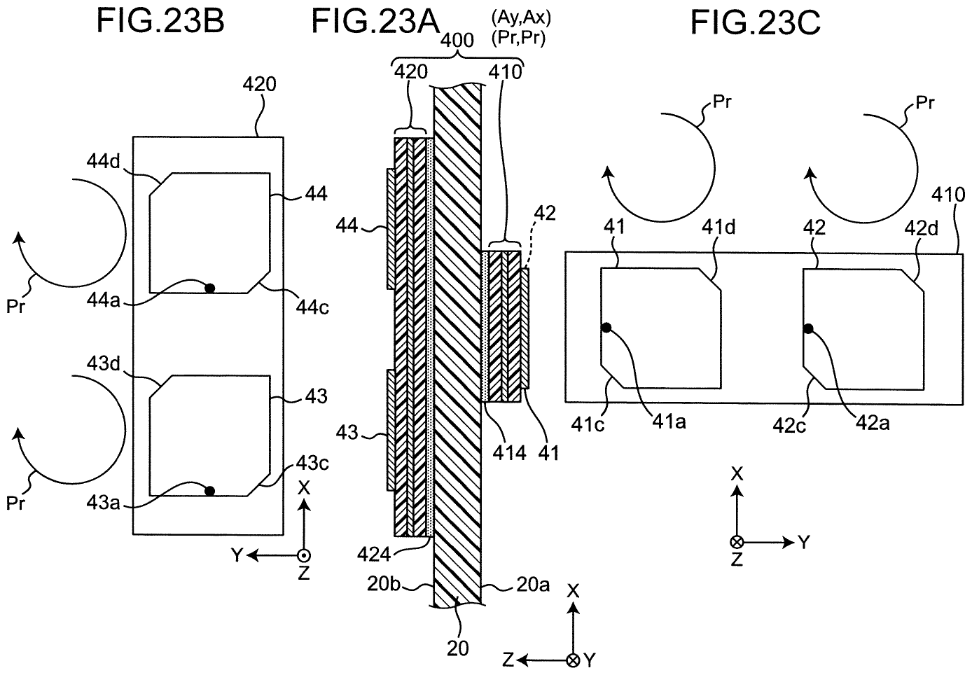

For the same reason as in the above example, for example, when the antenna arrangement (Ay, Ax) in which the first transmission/reception antenna pair (41, 42) are arranged side by side in the Y direction and the second transmission/reception antenna pair (43, 44) are arranged side by side in the X direction is employed as illustrated in FIG. 23A, in the first transmission antenna 41, the feed point 41a may be provided at the center of the side on the −Y side, the notch 41c may be provided at the corner formed by the side on the −X side and the side on the −Y side, and the notch 41d may be provided at the corner formed by the side on the +X side and the side on the +Y side as illustrated in FIG. 23C, so that the clockwise circular polarization Pr can be obtained as the polarization direction of the first transmission antenna 41. Furthermore, in the first reception antenna 42, the feed point 42a may be provided at the center of the side on the −Y side, the notch 42c may be provided at the corner formed by the side on the −X side and the side on the −Y side, and the notch 42d may be provided at the corner formed by the side on the +X side and the side on the +Y side, so that the clockwise circular polarization Pr can be obtained as the polarization direction of the first reception antenna 42. In this manner, the clockwise circular polarization Pr may be obtained as the polarization direction of the first transmission/reception antenna pair (41, 42). Similarly, in the second transmission antenna 43, the feed point 43a may be provided at the center of the side on the −X side, the notch 43c may be provided at the corner formed by the side on the −X side and the side on the −Y side, and the notch 43d may be provided at the corner formed by the side on the +X side and the side on the +Y side as illustrated in FIG. 23B, so that the clockwise circular polarization Pr can be obtained as the polarization direction of the second reception antenna 43. Furthermore, in the second reception antenna 44, the feed point 44a may be provided at the center of the side on the −X side, the notch 44c may be provided at the corner formed by the side on the −X side and the side on the −Y side, and the notch 44d may be provided at the corner formed by the side on the +X side and the side on the +Y side, so that the clockwise circular polarization Pr can be obtained as the polarization direction of the second reception antenna 44. In this manner, the clockwise circular polarization Pr may be obtained as the polarization direction of the second transmission/reception antenna pair (43, 44). In the upper portion of FIG. 23A, the combination of the polarization directions is represented by a sign (Pr, Pr), together with the sign (Ay, Ax) of the antenna arrangement.

### (Modification 4: Variation of Antenna Arrangement and Polarization Direction)

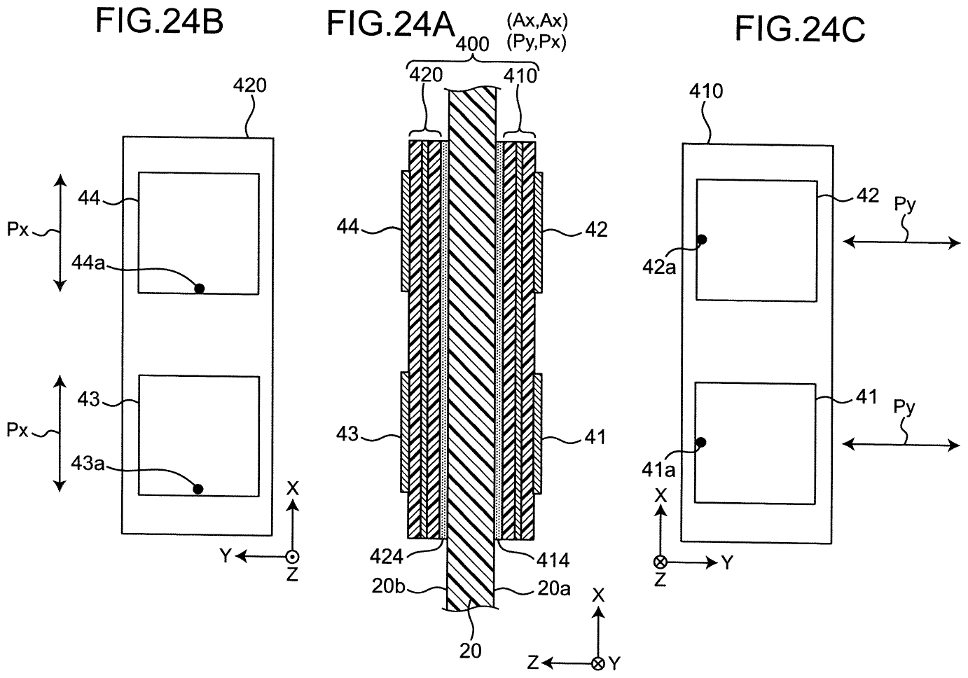

In the examples described above, the polarization direction of the first transmission/reception antenna pair (41, 42) is the same as the polarization direction of the second transmission/reception antenna pair (43, 44). However, the present invention is not limited to this. The polarization direction of the first transmission/reception antenna pair (41, 42) and the polarization direction of the second transmission/reception antenna pair (43, 44) may be different from each other. For example, when the antenna arrangement (Ax, Ax) in which the first transmission/reception antenna pair (41, 42) are arranged side by side in the X direction and the second transmission/reception antenna pair (43, 44) are arranged side by side in the X direction is employed as illustrated in FIG. 24A, in the first transmission antenna 41 and the first reception antenna 42, the feed points 41a and 42a may each be provided at the center of the side on the −Y side as illustrated in FIG. 24C to obtain the linear polarization Py as the polarization direction of the first transmission/reception antenna pair (41, 42). Furthermore in the second transmission antenna 43 and the second reception antenna 44, the feed points 43a and 44a may each be provided at the center of the side on the −X side as illustrated in FIG. 24B to obtain the linear polarization Px as the polarization direction of the second transmission/reception antenna pair (43, 44). Thus, the radio waves E1′ reflected by the radial artery 91 of the left wrist 90 and the radio waves E2′ reflected by the heart 81 can be distinguished from each other based on the polarization direction to be prevented from interfering. With such a setting, the pulse wave signal PS1 and the heartbeat signal PS2 can be acquired with high accuracy. In the upper portion of FIG. 24A, the combination of the polarization directions is represented by a sign (Py, Px), together with the sign (Ax, Ax) of the antenna arrangement.

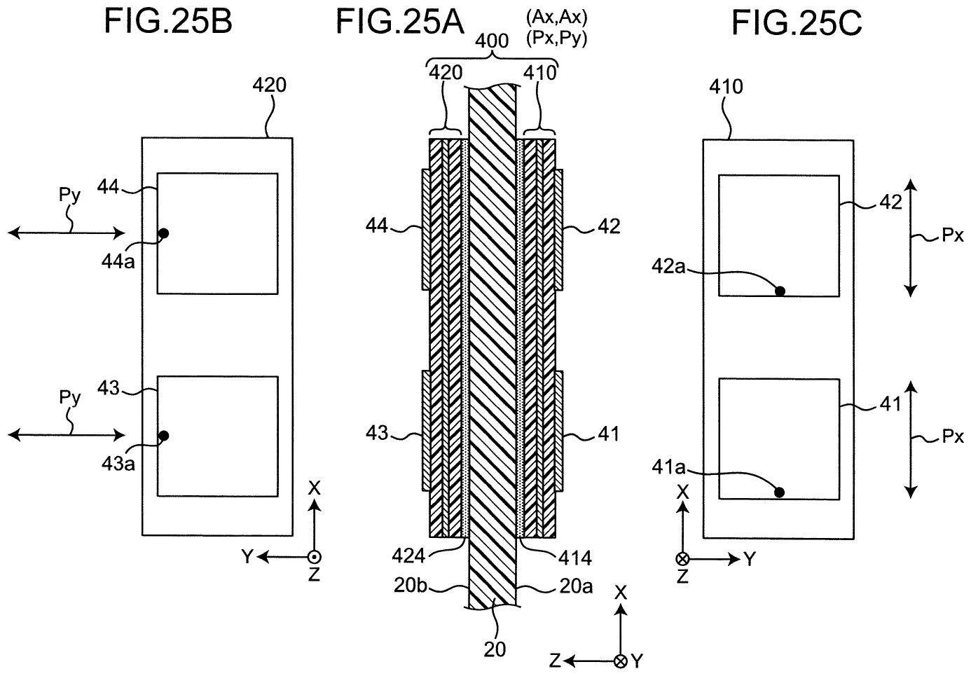

For the same reason as in the above example, for example, when the antenna arrangement (Ax, Ax) in which the first transmission/reception antenna pair (41, 42) are arranged side by side in the X direction and the second transmission/reception antenna pair (43, 44) are arranged side by side in the X direction is employed as illustrated in FIG. 25A, in the first transmission antenna 41 and the first reception antenna 42, the feed points 41a and 42a may each be provided at the center of the side on the −X side as illustrated in FIG. 25C to obtain the linear polarization Px as the polarization direction of the first transmission/reception antenna pair (41, 42). Furthermore in the second transmission antenna 43 and the second reception antenna 44, the feed points 43a and 44a may each be provided at the center of the side on the +Y side as illustrated in FIG. 25B to obtain the linear polarization Py as the polarization direction of the second transmission/reception antenna pair (43, 44). In the upper portion of FIG. 25A, the combination of the polarization directions is represented by a sign (Px, Py), together with the sign (Ax, Ax) of the antenna arrangement.

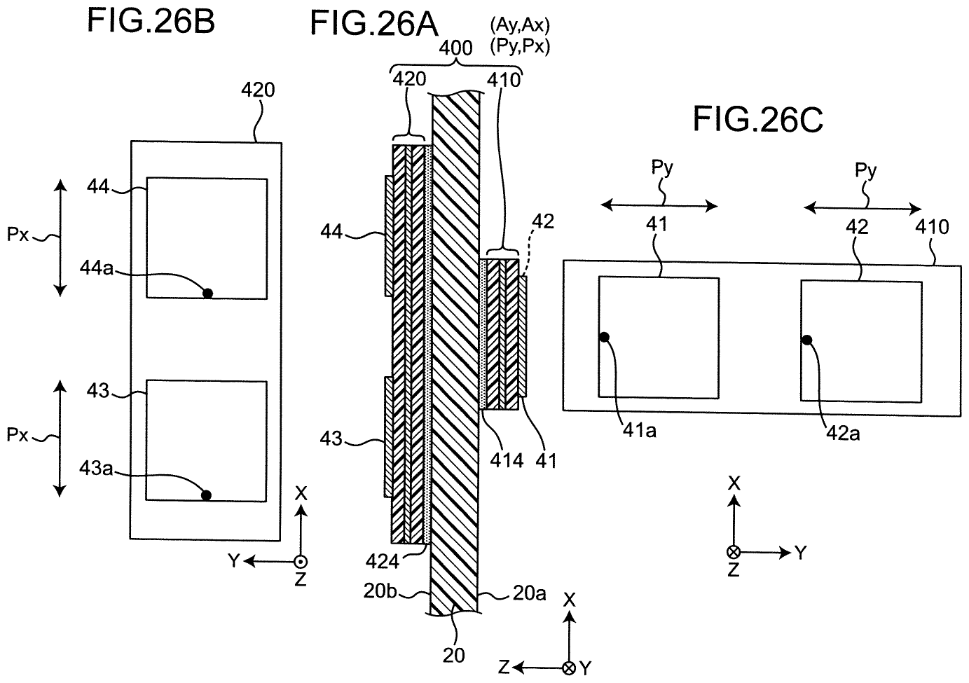

For the same reason as in the above example, for example, when the antenna arrangement (Ay, Ax) in which the first transmission/reception antenna pair (41, 42) are arranged side by side in the Y direction and the second transmission/reception antenna pair (43, 44) are arranged side by side in the X direction is employed as illustrated in FIG. 26A, in the first transmission antenna 41 and the first reception antenna 42, the feed points 41a and 42a may each be provided at the center of the side on the −Y side as illustrated in FIG. 26C to obtain the linear polarization Py as the polarization direction of the first transmission/reception antenna pair (41, 42). Furthermore in the second transmission antenna 43 and the second reception antenna 44, the feed points 43a and 44a may each be provided at the center of the side on the −X side as illustrated in FIG. 26B to obtain the linear polarization Px as the polarization direction of the second transmission/reception antenna pair (43, 44). In the upper portion of FIG. 26A, the combination of the polarization directions is represented by a sign (Py, Px), together with the sign (Ay, Ax) of the antenna arrangement.

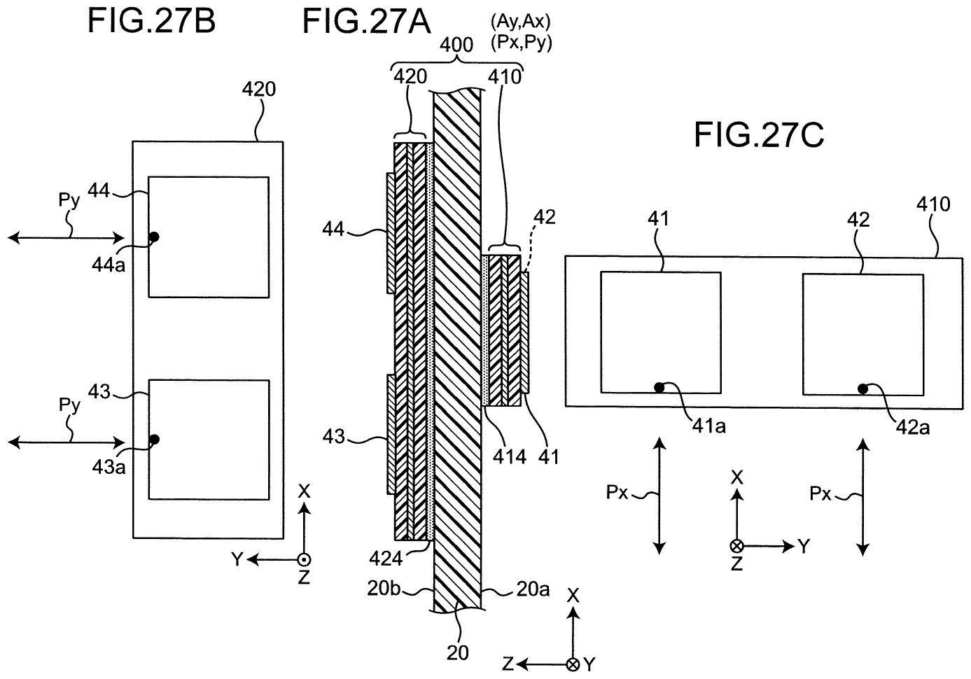

For the same reason as in the above example, for example, when the antenna arrangement (Ay, Ax) in which the first transmission/reception antenna pair (41, 42) are arranged side by side in the Y direction and the second transmission/reception antenna pair (43, 44) are arranged side by side in the X direction is employed as illustrated in FIG. 27A, in the first transmission antenna 41 and the first reception antenna 42, the feed points 41a and 42a may each be provided at the center of the side on the −X side as illustrated in FIG. 27C to obtain the linear polarization Px as the polarization direction of the first transmission/reception antenna pair (41, 42). Furthermore in the second transmission antenna 43 and the second reception antenna 44, the feed points 43a and 44a may each be provided at the center of the side on the +Y side as illustrated in FIG. 27B to obtain the linear polarization Py as the polarization direction of the second transmission/reception antenna pair (43, 44). In the upper portion of FIG. 27A, the combination of the polarization directions is represented by a sign (Px, Py), together with the sign (Ay, Ax) of the antenna arrangement.

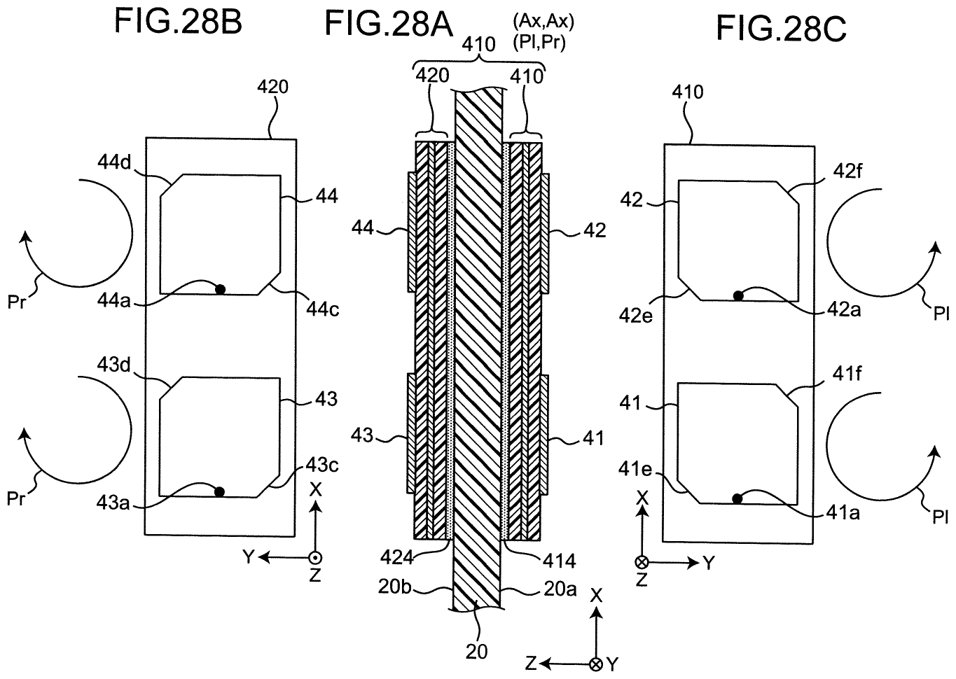

For the same reason as in the above example, for example, when the antenna arrangement (Ax, Ax) in which the first transmission/reception antenna pair (41, 42) are arranged side by side in the X direction and the second transmission/reception antenna pair (43, 44) are arranged side by side in the X direction is employed as illustrated in FIG. 28A, in the first transmission antenna 41, the feed point 41a may be provided at the center of the side on the −X side, a notch (perturbation element) 41e may be provided at the corner formed by the side on the −X side and the side on the −Y side, and a notch (perturbation element) 41f may be provided at the corner formed by the side on the +X side and the side on the +Y side as illustrated in FIG. 28C, so that counterclockwise circular polarization P1 can be obtained as the polarization direction of the first transmission antenna 41. Furthermore, in the first reception antenna 42, the feed point 42a may be provided at the center of the side on the −X side, a notch (perturbation element) 42e may be provided at the corner formed by the side on the −X side and the side on the −Y side, and a notch (perturbation element) 42f may be provided at the corner formed by the side on the +X side and the side on the +Y side, so that the counterclockwise circular polarization P1 can be obtained as the polarization direction of the first reception antenna 42. In this manner, the counterclockwise circular polarization P1 is obtained as the polarization direction of the first transmission/reception antenna pair (41, 42). On the other hand, in the second transmission antenna 43, the feed point 43a is provided at the center of the side on the −X side, the notch 43c is provided at the corner formed by the side on the −X side and the side on the −Y side, and the notch 43d is provided at the corner formed by the side on the +X side and the side on the +Y side as illustrated in FIG. 28B, so that the clockwise circular polarization Pr is obtained as the polarization direction of the second reception antenna 43. Furthermore, in the second reception antenna 44, the feed point 44a may be provided at the center of the side on the −X side, the notch 44c may be provided at the corner formed by the side on the −X side and the side on the −Y side, and the notch 44d may be provided at the corner formed by the side on the +X side and the side on the +Y side, so that the clockwise circular polarization Pr can be obtained as the polarization direction of the second reception antenna 44. In this manner, the clockwise circular polarization Pr different from the polarization direction (the counterclockwise circular polarization P1) of the first transmission/reception antenna pair (41, 42), is obtained as the polarization direction of the second transmission/reception antenna pair (43, 44). In the upper portion of FIG. 28A, the combination of the polarization directions is represented by a sign (P1, Pr), together with the sign (Ax, Ax) of the antenna arrangement.

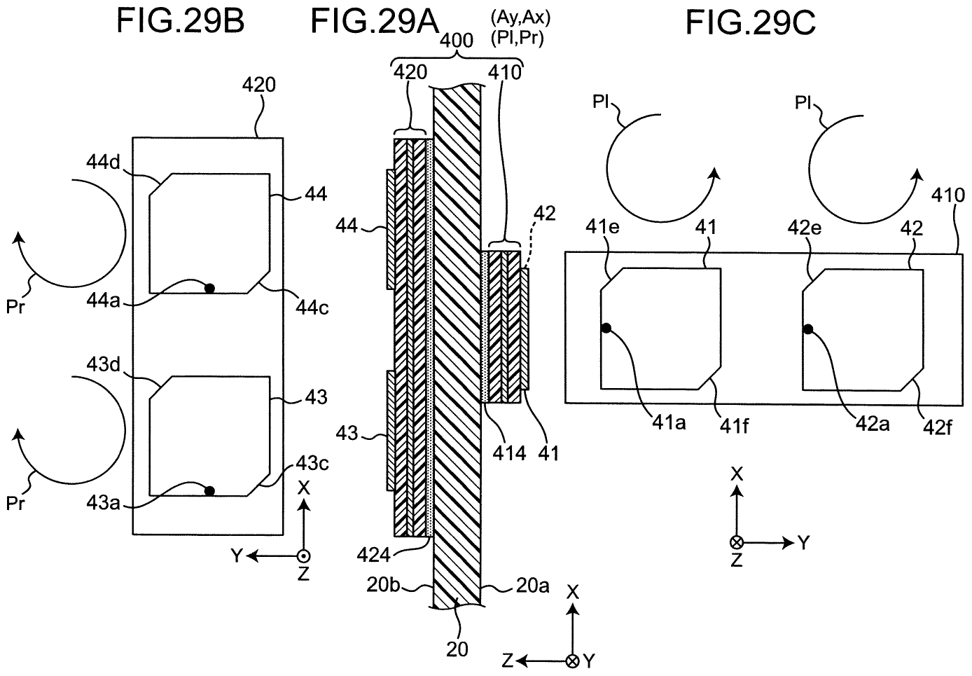

For the same reason as in the above example, for example, when the antenna arrangement (Ay, Ax) in which the first transmission/reception antenna pair (41, 42) are arranged side by side in the Y direction and the second transmission/reception antenna pair (43, 44) are arranged side by side in the X direction is employed as illustrated in FIG. 29A, in the first transmission antenna 41, the feed point 41a may be provided at the center of the side on the −Y side, the notch 41e may be provided at the corner formed by the side on the +X side and the side on the −Y side, and the notch 41f may be provided at the corner formed by the side on the −X side and the side on the +Y side as illustrated in FIG. 29C, so that counterclockwise circular polarization P1 can be obtained as the polarization direction of the first transmission antenna 41. Furthermore, in the first reception antenna 42, the feed point 42a may be provided at the center of the side on the −Y side, the notch 42e may be provided at the corner formed by the side on the +X side and the side on the −Y side, and the notch 42f may be provided at the corner formed by the side on the −X side and the side on the +Y side, so that the counterclockwise circular polarization P1 can be obtained as the polarization direction of the first reception antenna 42. In this manner, the counterclockwise circular polarization P1 is obtained as the polarization direction of the first transmission/reception antenna pair (41, 42). On the other hand, in the second transmission antenna 43, the feed point 43a is provided at the center of the side on the −X side, the notch 43c is provided at the corner formed by the side on the −X side and the side on the −Y side, and the notch 43d is provided at the corner formed by the side on the +X side and the side on the +Y side as illustrated in FIG. 29B, so that the clockwise circular polarization Pr is obtained as the polarization direction of the second reception antenna 43. Furthermore, in the second reception antenna 44, the feed point 44a may be provided at the center of the side on the −X side, the notch 44c may be provided at the corner formed by the side on the −X side and the side on the −Y side, and the notch 44d may be provided at the corner formed by the side on the +X side and the side on the +Y side, so that the clockwise circular polarization Pr can be obtained as the polarization direction of the second reception antenna 44. In this manner, the clockwise circular polarization Pr different from the polarization direction (the counterclockwise circular polarization P1) of the first transmission/reception antenna pair (41, 42), is obtained as the polarization direction of the second transmission/reception antenna pair (43, 44). In the upper portion of FIG. 29A, the combination of the polarization directions is represented by a sign (P1, Pr), together with the sign (Ay, Ax) of the antenna arrangement.

### (Modification 5; Integration of First Transmission Antenna and Second Transmission Antenna)

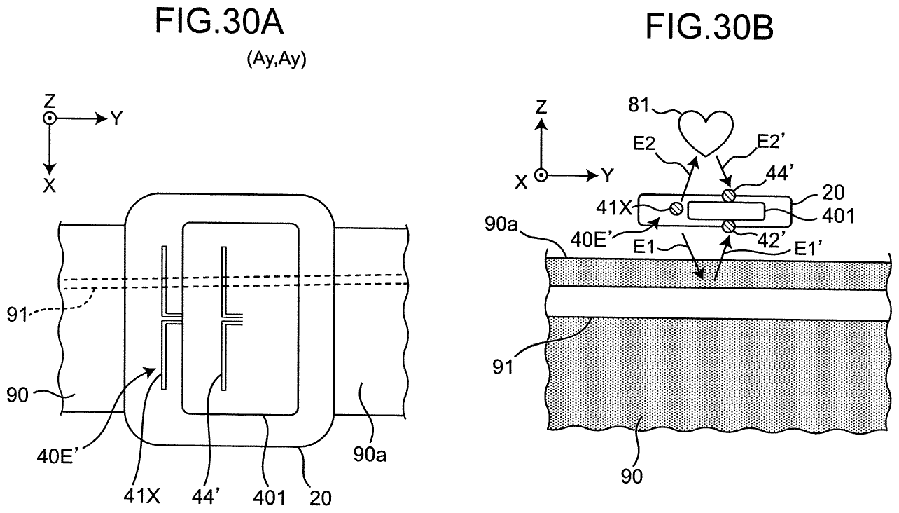

In the examples described above, for example, as illustrated in FIG. 3, the first transmission/reception antenna pair (41, 42) and the second transmission/reception antenna pair (43, 44) (in particular, the first transmission antenna 41 and the second transmission antenna 43) are respectively attached to the inner circumferential surface 20a and the outer circumferential surface 20b of the belt 20, via the base section 400 (including the substrates 410 and 420). However, the present invention is not limited to this. For example, instead of the above-described first transmission antenna 41 and second transmission antenna 43, a common third transmission antenna 41X may be provided that emits the radio waves E1 and E2 to both of the radial artery 91 of the left wrist 90 and the heart 81 as illustrated in FIGS. 30A and 30B. Here, FIG. 30A illustrates a planar layout of a transmission and reception antenna group (denoted with a sign 40E) according to this modification, in a state where the belt 20 is worn around the left wrist 90. FIG. 30B schematically illustrates a cross section along the longitudinal direction (Y direction) of left wrist 90 in FIG. 30A. As illustrated in these figures, in this example, a small base section 401 having a shorter dimension in the Y direction than the above-described base section 400, as well as a third transmission antenna 41X, a first reception antenna 42′, and a second reception antenna 44′ included in the transmission and reception antenna group 40E′ are provided. Each of the third transmission antenna 41X, the first reception antenna 42′, and the second reception antenna 44′ is formed by a dipole antenna extending in the X direction in this example. The third transmission antenna 41X is positioned adjacent to the base section 401 while being separated therefrom toward the −Y side. The first reception antenna 42′ is arranged along the inner circumferential surface 20a of the belt 20 (the base section 401). The second reception antenna 44′ is arranged along the outer circumferential surface 20b of the belt 20 (the base section 401). Thus, in the worn state, as illustrated in FIG. 30B, the third transmission antenna 41X faces both the radial artery 91 of left wrist 90 and the heart 81. The first reception antenna 42′ faces the radial artery 91 in the left wrist 90, and the second reception antenna 44′ faces the heart 81. In this example, the third transmission antenna 41X and the first reception antenna 42 form a first transmission/reception antenna pair (41X, 42′), and the third transmission antenna 41X and the second reception antenna 44′ form a second transmission/reception antenna pair (41X, 44′). The first transmission/reception antenna pair (41X, 42′) and the second transmission/reception antenna pair (41X, 44′) are each arranged side by side in the Y direction. Thus, this antenna arrangement is represented by a sign (Ay, Ay) in the upper part of FIG. 30A.

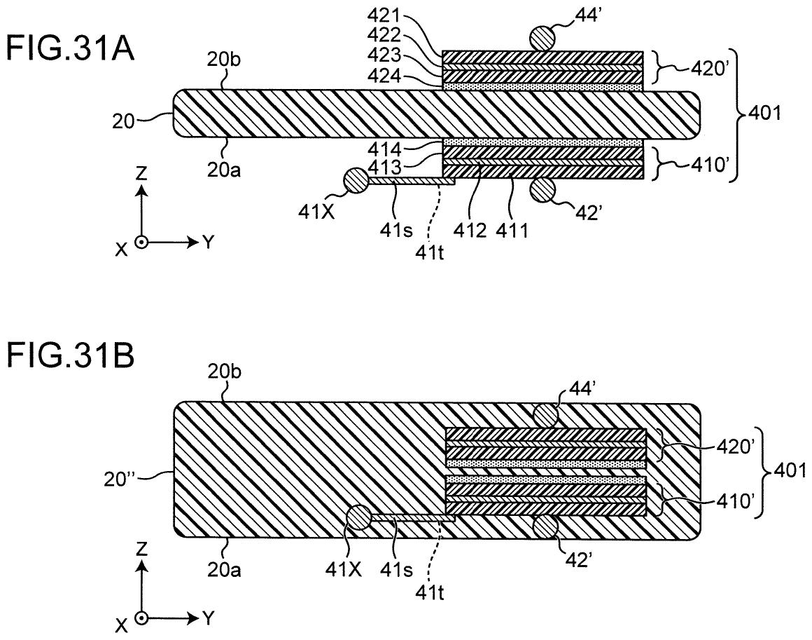

FIG. 31A illustrates a cross-sectional structure of the transmission and reception antenna group 40E′. In this example, the first reception antenna 42′ and the second reception antenna 44′ are respectively provided to the inner circumferential surface 20a and the outer circumferential surface 20b of the belt 20 via substrates 410′ and 420′. The substrates 410′ and 420′ have the same cross-sectional structure as the substrates 410 and 420 described above, but have a smaller dimension in the Y direction than these counterparts. In this example, the third transmission antenna 41X is arranged along the inner circumferential surface 20a of the belt 20. The third transmission antenna 41X and the substrate 410′ are connected to each other by feeders 41s and 41t. Note that the third transmission antenna 41X may be arranged along the outer circumferential surface 20b of the belt 20, and may be connected to the substrate 420′ via the feeders 41s and 41t. As illustrated in FIG. 31B, a belt (denoted with a reference numeral 20″) may be obtained by embedding each of the third transmission antenna 41X, the first reception antenna 42′, and the second reception antenna 44′ in the belt 20 so that the belt becomes flat on the inner circumferential surface 20a and on the outer circumferential surface 20b. In this example, the thickness of the belt 20″ is set to 8 mm. In this case, since the inner circumferential surface 20a side of the belt 20″ is flat, the subject 80 does not feel uncomfortable wearing the belt 20″ (which the subject 80 may feel if the belt has recesses and protrusions on the inner circumferential surface side), as in the case described with reference to FIG. 6B. Furthermore, with the outer circumferential surface 20b side of the belt 20″ is flat, the transmission and reception antenna group 40E′ of the sphygmomanometer 1 is less likely to break even when the outer circumferential surface 20b of the belt 20″ comes into contact with a desk, a wall, or the like due to the activity of the subject 80. Furthermore, a better appearance can be achieved.

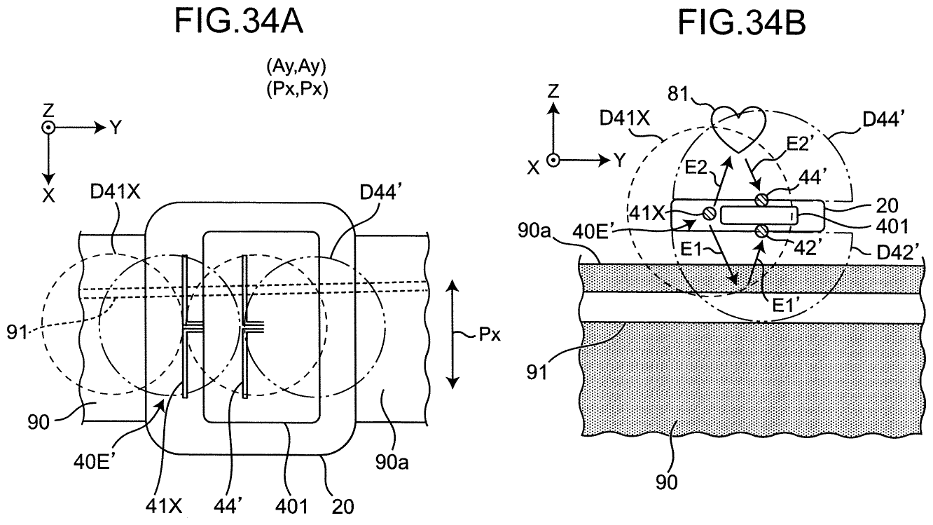

As illustrated in FIG. 33, each dipole antenna (in this example, the third transmission antenna 41X is representatively illustrated) includes a pair of elements 41Xa and 41Xb extending linearly in opposite directions. The length of each of the elements 41Xa and 41Xb is set to be about ¼ wavelength of the used frequency. During operation, power is fed to the location where the elements 41Xa and 41Xb are closest to each other, through the feeder 41s and 41t. As illustrated in FIG. 33, each dipole antenna corresponds to the linear polarization Px along the direction in which the elements 41Xa and 41Xb extend (the X direction in this example). Thus, the polarization direction of each of the first transmission/reception antenna pair (41X, 42′) and the second transmission/reception antenna pair (41X, 44′) is the linear polarization Px. In the upper portion of FIG. 34A, the combination of the polarization directions is represented by a sign (Px, Px), together with the sign (Ay, Ay) of the antenna arrangement. FIGS. 34A and 34B illustrate configurations obtained by additionally indicating the directivity and the polarization direction in FIGS. 30A and 30B, respectively.

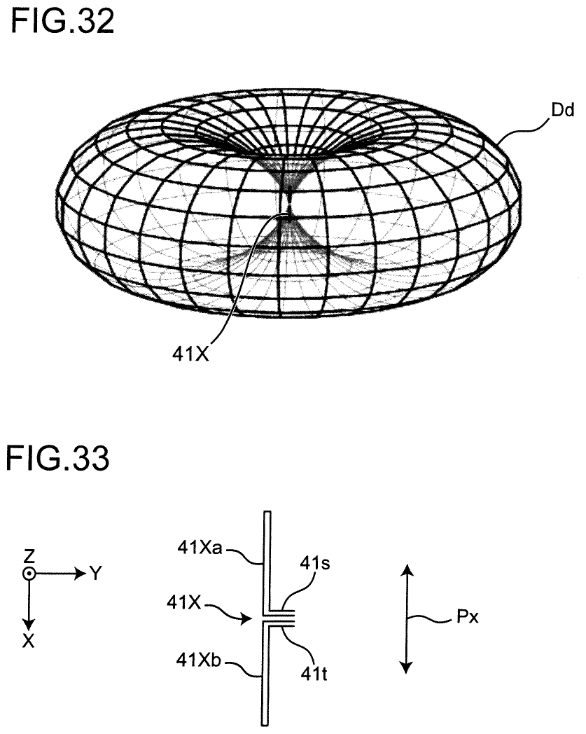

Furthermore, as illustrated in FIG. 32, each dipole antenna has a circular directivity in a plane perpendicular to the elements 41Xa and 41Xb, and has an infinity mark shaped directivity in a plane including the elements 41Xa and 41Xb. Accordingly, in the XY plane illustrated in FIG. 34A, the third transmission antenna 41X has a directivity D41X in an infinity mark shape elongated in the X direction as indicated by the broken line. The second reception antenna 44′ also has a directivity D44′ in an infinity mark shape elongated in the X direction as indicated by a two-dot chain line. In the YZ plane illustrated in FIG. 34B, the third transmission antenna 41X has a circular directivity D41X indicated by a broken line. The first reception antenna 42 is shielded by the base section 401 (including the copper layers 412 and 422 as shielding layers) and has a semicircular directivity (spreading in the −Z direction) D42′ indicated by a two-dot chain line. Similarly, the second reception antenna 44′ is shielded by the base section 401 and has a semicircular directivity (spreading in the +Z direction) D44′ indicated by a two-dot chain line.

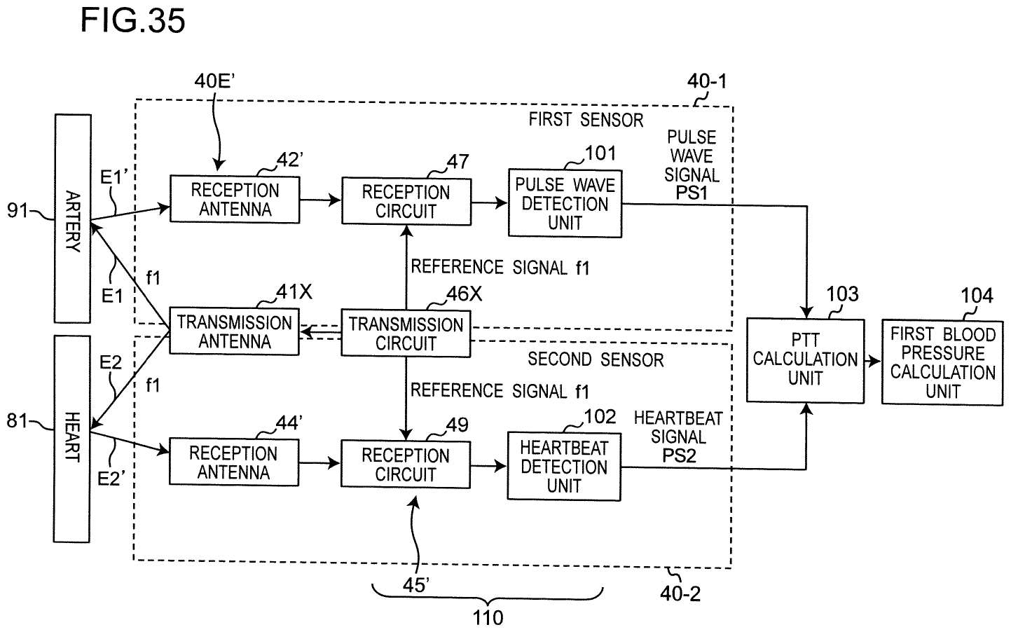

In this example, as illustrated in FIG. 35, the transmission and reception circuit group 45′ of the transmission and reception unit 40 includes the transmission circuit 46X connected to the third transmission antenna 41X, and the reception circuits 47 and 49 respectively connected to the reception antennas 42′ and 44′. The transmission circuit 46X under operation emits the radio waves E1 and E2 at a frequency f1 (f1=24.05 GHz in this example) in a 24 GHz band in this example, respectively toward the radial artery 91 and the heart 81 (actually, emitted in isometric directions in the YZ plane as illustrated in FIG. 34B) through the third transmission antenna 41X. At the same time, the reception circuit 47 identifies the radio waves E1′ reflected by the radial artery 91 of the left wrist 90 based on the reference signal (frequency f1) from the transmission circuit 46X, receives the waves via the first reception antenna 42′, and detects and amplifies the waves. On the other hand, the reception circuit 49 identifies the radio waves E2′ reflected by the heart 81 based on the reference signal (frequency f1) from the transmission circuit 46X, receives the waves via the second reception antenna 44′, and detects and amplifies the waves. In this example, with the transmission and reception circuit group 45′ provided in the transmission and reception unit 40, a power feeding path from the transmission circuit 46X to the third transmission antenna 41X can be made relatively short, where by degradation of the waveforms of the radio waves E1 and E2 can be suppressed. Furthermore, a reception path from the respective reception antennas 42′ and 44′ to the reception circuits 47 and 49 can be made relatively short. Further, with the base section 401 (including the copper layers 412 and 422 as the shielding layers) shielding radio waves between the first reception antenna 42′ and the second reception antenna 44′, interference between the pulse wave signal PS1 and the heartbeat signal PS2 is suppressed. As a result, the pulse wave signal PS1 and the heartbeat signal PS2 can be acquired with high accuracy.

Furthermore, in this example, since the third transmission antenna 41X serving as both the first transmission antenna 41 and the second transmission antenna 43 described above is provided, the configuration of the sphygmomanometer 1 can be simplified.

### (Modification 6: Variation in Frequency)

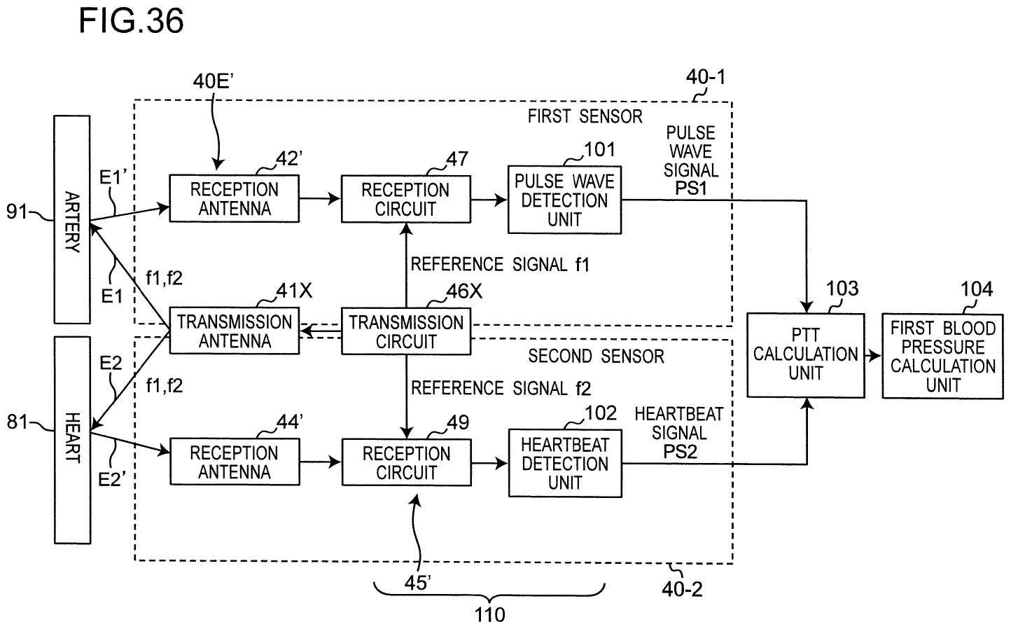

In the above example, as illustrated in FIG. 35, the frequency of the radio wave E1′ received by the reception circuit 47 and the frequency of the radio wave E2′ received by the reception circuit 49 are the same frequency f1. However, the present invention is not limited to this. For example, as illustrated in FIG. 36, the third transmission antenna 41X emits the radio waves E1, including a first frequency component f1 and a second frequency component f2 different from each other, toward the radial artery 91 in the left wrist 90, and also emit the radio waves E2 including the first frequency component f1 and the second frequency component f2 different from each other toward the heart 81. In this example, f1=24.05 GHz and f2=24.25 GHz. Furthermore, the reception circuit 47 identifies a component corresponding to the first frequency component f1 in the radio waves E1′ reflected by the radial artery 91 of the left wrist 90 based on the reference signal (frequency f1) from the transmission circuit 46X, receives the waves via the first reception antenna 42′, and detects and amplifies the waves. On the other hand, the reception circuit 49 identifies a component corresponding to the second frequency component f2 in the radio waves E2′ reflected by the heart 81 based on the reference signal (frequency f2) from the transmission circuit 46X, receives the waves via the second reception antenna 44′, and detects and amplifies the waves.

In this case, the radio waves E1′ reflected by the radial artery 91 of the left wrist 90 and the radio waves E2′ reflected by the heart 81 can be distinguishable from each other based on the frequencies f1 and f2 to be prevented from interfering. As a result, the pulse wave signal PS1 and the heartbeat signal PS2 can be acquired with even higher accuracy.

### (Modification 7: Variation of Antenna Arrangement and Polarization Direction)

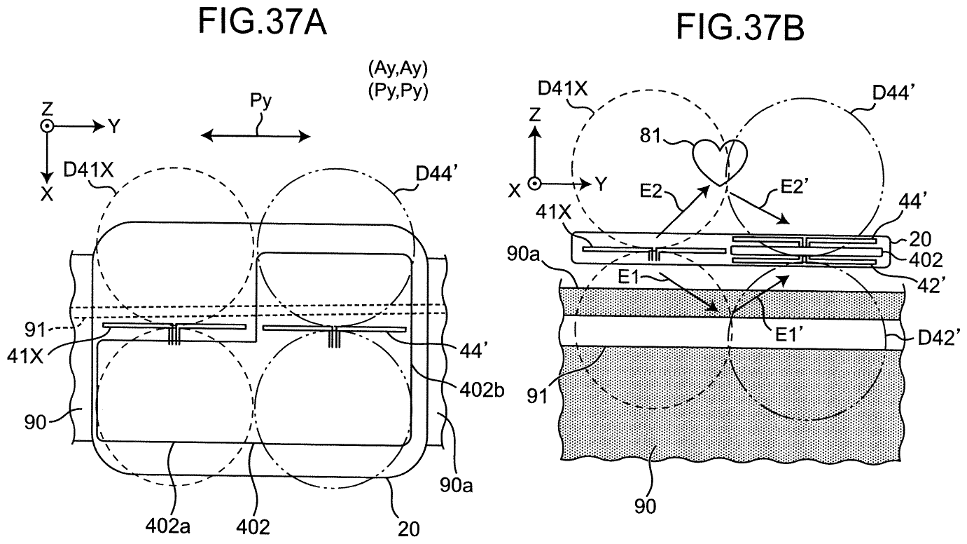

In the above example, for example, when the antenna arrangement (Ay, Ay) in which the first transmission/reception antenna pair (41X, 42′) are arranged side by side in the Y direction and the second transmission/reception antenna pair (41X, 44′) are arranged side by side in the Y direction is employed as illustrated in FIGS. 34A and 34B, the polarization directions of the first transmission/reception antenna pair (41X, 42′) and of the second transmission/reception antenna pair (41X, 44′) are both the linear polarization Px. However, the present invention is not limited to this. For example, when the antenna arrangement (Ay, Ay) in which the first transmission/reception antenna pair (41X, 42′) are arranged side by side in the Y direction and the second transmission/reception antenna pair (41X, 44′) are arranged side by side in the Y direction is employed as illustrated in FIGS. 37A and 37B, the polarization directions of the first transmission/reception antenna pair (41X, 42′) and of the second transmission/reception antenna pair (41X, 44′) may both be the linear polarization Py. In this example, a base section 402 is provided instead of the above-described base section 401. As illustrated in FIG. 37A, this base section 402 has a straight portion 402a extending in the Y direction on the XY plane, and a straight portion 402b connected to the straight portion 402a and extending in the X direction, and thus has a substantially L-shaped planar shape. The cross-sectional structure of the base section 402 is the same as the cross-sectional structure of the base section 401. The third transmission antenna 41X is arranged so as to extend in the Y direction at a position outside the base section 402 and in a recessed part of the L shape. The first reception antenna 42′ is arranged along the inner circumferential surface 20a of the belt 20 (the base section 402) to extend in the Y direction, as illustrated in FIG. 37B. The second reception antenna 44′ is arranged along the outer circumferential surface 20b of the belt 20 (the base section 402) to extend in the Y direction. Thus, the polarization direction of each of the first transmission/reception antenna pair (41X, 42′) and the second transmission/reception antenna pair (41X, 44′) is the linear polarization Py. In the upper portion of FIG. 37A, the combination of the polarization directions is represented by a sign (Py, Py), together with the sign (Ay, Ay) of the antenna arrangement. In this example, in the XY plane illustrated in FIG. 37A, the third transmission antenna 41X has a directivity D41X in an infinity mark shape elongated in the X direction as indicated by the broken line. The second reception antenna 44′ also has a directivity D44′ in an infinity mark shape elongated in the X direction as indicated by a two-dot chain line (the same applies to the first reception antenna 42′). In the YZ plane illustrated in FIG. 37B, the third transmission antenna 41X has a directivity D41X in an infinity mark shape elongated in the X direction as indicated by the broken line. The first reception antenna 42′ is shielded by the base section 402 (including the copper layers 412 and 422 as shielding layers) and has a circular directivity (spreading in the −Z direction) D42′ indicated by a two-dot chain line. Similarly, the second reception antenna 44′ is shielded by the base section 402 and has a circular directivity (spreading in the +Z direction) D44′ indicated by a two-dot chain line. In the worn state, as illustrated in FIG. 37B, the third transmission antenna 41X faces both the radial artery 91 of left wrist 90 and the heart 81. The first reception antenna 42′ faces the radial artery 91 in the left wrist 90, and the second reception antenna 44′ faces the heart 81. Therefore, during operation, the radio waves E1 and E2 can be emitted toward both the radial artery 91 of the left wrist 90 and the heart 81, and the radio waves E1′ reflected by the radial artery 91 and the radio waves E2′ reflected by the heart 81 can be received.

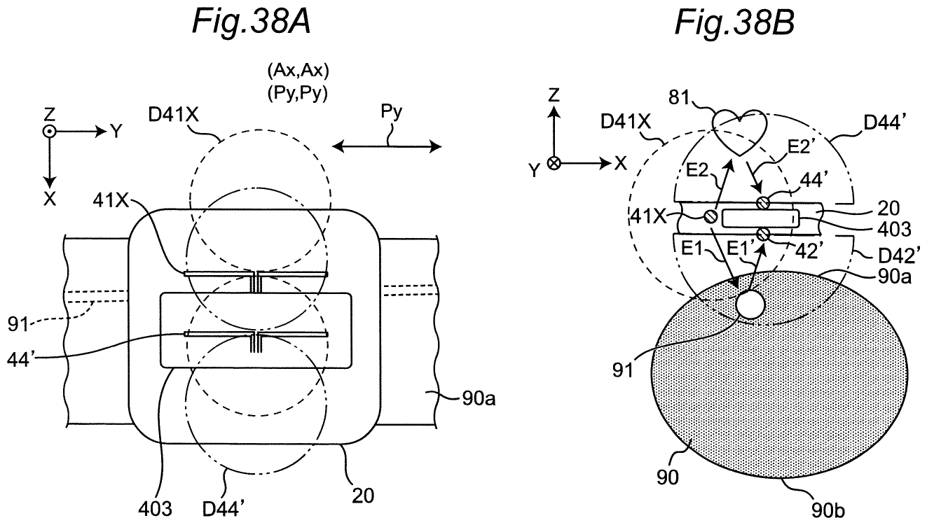

For example, when the antenna arrangement (Ax, Ax) in which the first transmission/reception antenna pair (41X, 42′) are arranged side by side in the X direction and the second transmission/reception antenna pair (41X, 44′) are arranged side by side in the X direction is employed as illustrated in FIGS. 38A and 38B, the polarization directions of the first transmission/reception antenna pair (41X, 42′) and of the second transmission/reception antenna pair (41X, 44′) may both be the linear polarization Py. In this example, a small base section 403 is provided instead of the above-described base section 401. As illustrated in FIG. 38A, in the XY plane, the base section 403 has a shorter dimension in the X direction and a longer dimension in the Y direction compared with the base section 401. The cross-sectional structure of the base section 403 is the same as the cross-sectional structure of the base section 401. The third transmission antenna 41X is positioned adjacent to the base section 403 while being separated therefrom toward the −X side. The first reception antenna 42′ is arranged along the inner circumferential surface 20a of the belt 20 (the base section 403) to extend in the Y direction, as illustrated in FIG. 38B. The second reception antenna 44′ is arranged along the outer circumferential surface 20b of the belt 20 (the base section 403) to extend in the Y direction. Thus, the polarization direction of each of the first transmission/reception antenna pair (41X, 42′) and the second transmission/reception antenna pair (41X, 44′) is the linear polarization Py. In the upper portion of FIG. 38A, the combination of the polarization directions is represented by a sign (Py, Py), together with the sign (Ax, Ax) of the antenna arrangement. In this example, in the XY plane illustrated in FIG. 38A, the third transmission antenna 41X has a directivity D41X in an infinity mark shape elongated in the X direction as indicated by the broken line. The second reception antenna 44′ also has a directivity D44′ in an infinity mark shape elongated in the X direction as indicated by a two-dot chain line (the same applies to the first reception antenna 42′). In the ZX plane illustrated in FIG. 38B, the third transmission antenna 41X has a circular directivity D41X indicated by a broken line. The first reception antenna 42′ is shielded by the base section 403 (including the copper layers 412 and 422 as shielding layers) and has a semicircular directivity (spreading in the −Z direction) D42′ indicated by a two-dot chain line. Similarly, the second reception antenna 44′ is shielded by the base section 403 and has a semicircular directivity (spreading in the +Z direction) D44′ indicated by a two-dot chain line. As in the example described above, in the worn state, as illustrated in FIG. 38B, the third transmission antenna 41X faces both the radial artery 91 of left wrist 90 and the heart 81. The first reception antenna 42′ faces the radial artery 91 in the left wrist 90, and the second reception antenna 44′ faces the heart 81. Therefore, during operation, the radio waves E1 and E2 can be emitted toward both the radial artery 91 of the left wrist 90 and the heart 81, and the radio waves E1′ reflected by the radial artery 91 and the radio waves E2′ reflected by the heart 81 can be received.

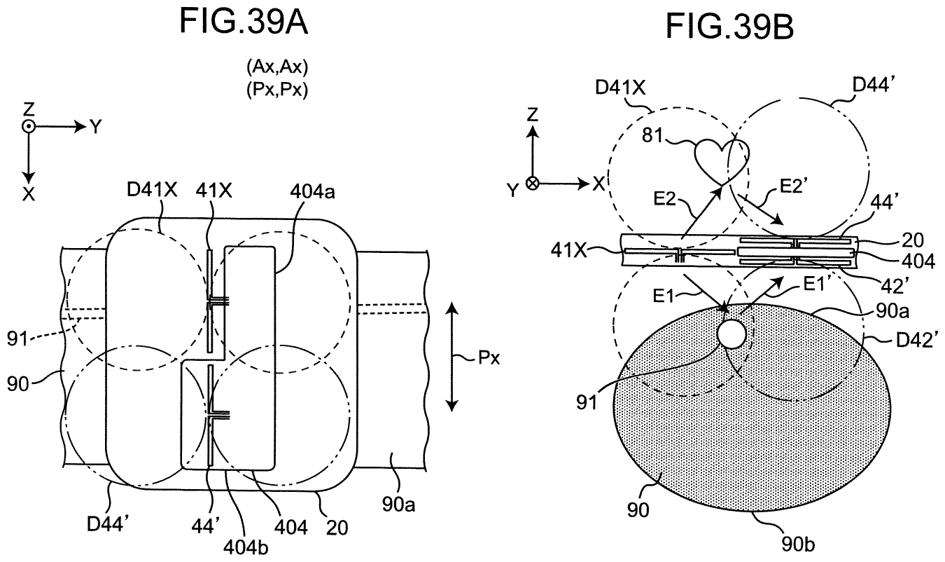

For example, when the antenna arrangement (Ax, Ax) in which the first transmission/reception antenna pair (41X, 42′) are arranged side by side in the X direction and the second transmission/reception antenna pair (41X, 44′) are arranged side by side in the X direction is employed as illustrated in FIGS. 39A and 39B, the polarization directions of the first transmission/reception antenna pair (41X, 42′) and of the second transmission/reception antenna pair (41X, 44′) may both be the linear polarization Px. In this example, a base section 404 is provided instead of the above-described base section 401. As illustrated in FIG. 39A, this base section 404 has a straight portion 404a extending in the X direction on the XY plane, and a straight portion 404b connected to the straight portion 404a and extending in the Y direction, and thus has a substantially L-shaped planar shape. The cross-sectional structure of the base section 404 is the same as the cross-sectional structure of the base section 401. The third transmission antenna 41X is arranged so as to extend in the X direction at a position outside the base section 404 and in a recessed part of the L shape. The first reception antenna 42′ is arranged along the inner circumferential surface 20a of the belt 20 (the base section 404) to extend in the X direction, as illustrated in FIG. 39B. The second reception antenna 44′ is arranged along the outer circumferential surface 20b of the belt 20 (the base section 404) to extend in the X direction. Thus, the polarization direction of each of the first transmission/reception antenna pair (41X, 42′) and the second transmission/reception antenna pair (41X, 44) is the linear polarization Px. In the upper portion of FIG. 39A, the combination of the polarization directions is represented by a sign (Px, Px), together with the sign (Ax, Ax) of the antenna arrangement. In this example, in the XY plane illustrated in FIG. 39A, the third transmission antenna 41X has a directivity D41X in an infinity shape elongated in the Y direction indicated by a broken line. The second reception antenna 44′ also has a directivity D44′ in an infinity mark shape elongated in the Y direction as indicated by a two-dot chain line (the same applies to the first reception antenna 42′). In the ZX plane illustrated in FIG. 39B, the third transmission antenna 41X has a directivity D41X in an infinity mark shape elongated in the Z direction as indicated by the broken line. The first reception antenna 42′ is shielded by the base section 403 (including the copper layers 412 and 422 as shielding layers) and has a circular directivity (spreading in the −Z direction) D42′ indicated by a two-dot chain line. Similarly, the second reception antenna 44′ is shielded by the base section 402 and has a circular directivity (spreading in the +Z direction) D44′ indicated by a two-dot chain line. As in the example described above, in the worn state, as illustrated in FIG. 39B, the third transmission antenna 41X faces both the radial artery 91 of left wrist 90 and the heart 81. The first reception antenna 42′ faces the radial artery 91 in the left wrist 90, and the second reception antenna 44′ faces the heart 81. Therefore, during operation, the radio waves E1 and E2 can be emitted toward both the radial artery 91 of the left wrist 90 and the heart 81, and the radio waves E1′ reflected by the radial artery 91 and the radio waves E2′ reflected by the heart 81 can be received.

### (Variation in Measurement Target Part)

In the examples described above, it is assumed that the sphygmomanometer 1 is expected to be worn around the left wrist 90 that is the measurement target part. However, the present invention is not limited to this. The measurement target part may be any part where the artery runs, and may be a right wrist, or an upper limb part other than the wrist such as an upper arm, a forearm, a hand, or a finger.

For example, with reference to FIG. 1, when the measurement target part is the upper arm, the subject 80 wears the belt 20 with the transmission and reception unit 40 provided on the inner side of the upper arm (trunk 82 side), and takes the “recommended measurement posture” with the upper extending along the side of the trunk 82. As a result, the first transmission/reception antenna pair (41, 42) face the artery 91 of the upper arm, and the second transmission/reception antenna pair (43, 44) face the heart 81. Also in the operation in such a state, the distance between the first transmission/reception antenna pair (41, 42) and the artery 91 in the upper arm is expected to be about 5 mm, and the distance between the second transmission/reception antenna pair (43, 44) and the heart 81 is assumed to be about 50 mm. Based on these distances, the intensity levels of the radio waves emitted by the first transmission antenna 41 and the second transmission antenna 43 are about 0.5 mW and about 10 mW, respectively. The reception levels of the reception antennas 42 and 44 are about 1 μW and about 0.2 μW, respectively. The output level of each of the reception circuits 47 and 49 is about 1 volt. Furthermore, the intensity levels at peaks A1 and A2 of the pulse wave signal PS1 and the heartbeat signal PS2 are each about 100 mV to 1 volt. With such a setting, the pulse wave signal PS1 and the heartbeat signal PS2 can be acquired with high accuracy.

### (Variation in Control System)

Furthermore, in the examples described above, the CPU 100 provided to the sphygmomanometer 1 functions as the pulse wave detection unit 101, the heartbeat detection unit 102, the PTT calculation unit 103, and the first and the second blood pressure calculation units 104 and 204, to execute the blood pressure measurement through the oscillometric method (the operation flow in FIG. 11) and the blood pressure measurement (estimation) based on the PTT (the operation flow in FIG. 14). However, the present invention is not limited to this. For example, a substantial computer device such as a smartphone provided outside the sphygmomanometer 1 may function as the pulse wave detection unit 101, the heartbeat detection unit 102, the PTT calculation unit 103, and the first and the second blood pressure calculation units 104 and 204, and cause the sphygmomanometer 1 to execute the blood pressure measurement by the oscillometric method (the operation flow in FIG. 11) and the blood pressure measurement (estimation) based on the PTT (the operation flow in FIG. 14), through the network 900. In this case, the user performs an operation such as an instruction to start or stop the blood pressure measurement using an operation unit (such as a touch panel, a keyboard, or a mouse) of the computer device, and cause a display (such as an organic EL display or an LCD) of the computer device to display information related to the blood pressure measurement such as a blood pressure measurement result and other types of information. In that case, the display 50 and the operation unit 52 may be omitted in the sphygmomanometer 1.

In addition, the sphygmomanometer 1 or the computer device may include a timer capable of setting a measurement time in advance. Thus, when the current time reaches (or approaches) the measurement time set in the timer, the subject may be notified of such a fact through display or sound prompting the user to take the recommended measurement posture. Unless the user takes the recommended measurement posture, the sphygmomanometer 1 or the computer may not operate (may not perform the pulse wave measurement) or may cause only the pulse wave detection unit to operate without performing the blood pressure measurement (estimation).

### (Variation in Vital Sign)

In the above-described example, the pulse wave signal, the heartbeat signal, the PTT, and the blood pressure are measured as the vital sign by the sphygmomanometer 1. However, the present invention is not limited to this. Various other types of vital sign such as a pulse wave rate may be measured.

### (Variation as Apparatus)

Furthermore, according to the present invention, an apparatus including the vital sign measurement device and/or the blood pressure measurement device and further including a functional unit for executing further function may be configured. With this apparatus, vital sign can be accurately measured, and particularly, a pulse wave signal and a heartbeat signal can be accurately obtained as the vital sign, or a blood pressure value can be accurately calculated (estimated). This apparatus can perform various further functions.

As described above, a vital sign measurement device of the present disclosure is a vital sign measurement device that measures a pulse wave of an artery and a heartbeat of a heart of a living body, the vital sign measurement device comprising:

a belt to be worn around an upper limb part of the living body; and

a transmission and reception unit that is capable of transmitting and receiving radio waves, the transmission and reception unit being provided at a portion of the belt to face both an artery running in the upper limb part and the heart when the living body takes a predetermined recommended measurement posture in a worn state of the belt being worn around the upper limb part, wherein

the transmission and reception unit includes:

a transmission antenna unit that emits radio waves to each of the artery in the upper limb part and the heart; and

a reception antenna unit that receives radio waves reflected by the artery in the upper limb part and/or a tissue being displaced in accordance with a pulse wave of the artery and by the heart and/or a tissue being displaced in accordance with the heartbeat of the heart, and

the vital sign measurement device further comprises a vital sign detection unit that acquires a pulse wave signal representing the pulse wave of the artery in the upper limb part and a heartbeat signal representing the heartbeat of the heart based on an output from the reception antenna unit.

As used herein, the “upper limb part” includes the upper arms, the forearms, the hands, and the fingers.

The portion of the belt at which the transmission and reception unit is mounted is set in advance as a portion facing both the artery running in the upper limb part and the heart, when the living body takes a predetermined “recommended measurement posture” in a state where the belt is worn around the upper limb part. The term “facing” may indicate any state where the radio waves can be transmitted and received to and from each other, between the transmission and reception unit and the upper limb part, and between the transmission and reception unit and the heart. Thus, facing each other indirectly with clothes and the like provided therebetween is included.

As the “recommended measurement posture”, a posture where the artery in the upper limb part and the heart are (almost) at the same height, with respect to the direction of gravitational acceleration or the like, is recommended. For example, when the upper limb part is an upper arm, a posture with the upper arm extending along a side of the trunk may be employed. Alternatively, when the upper limb part is a wrist, the following “recommended measurement posture” may be employed in a state where the living body stands straight. Specifically, a subject raises his or her forearm so that the forearm diagonally extends (hand up, elbow down) in front of and while overlapping with the trunk. The wrist is maintained at the same height level as the heart. The palm side surface of the wrist (a part of the outer circumferential surface of the wrist corresponding to the palm) faces the heart. When the upper limb part is the wrist and the living body is lying on his/her back, the posture with the wrist put on the front chest is not recommended.

The “tissue being displaced in accordance with the pulse wave of the artery” of the upper limb part is a portion of the living body that is displaced in accordance with the pulse wave of the artery (causing the expansion and contraction of blood vessels). For example, in a “skin-fatty layer-artery” configuration, a skin of the upper limb part is included. The “tissue being displaced in accordance with the heartbeat of the heart” is a portion of the living body that is displaced in accordance with the heartbeat of the heart.

In a vital sign measurement device according to this disclosure, a belt is worn around an upper limb part of a living body. When the living body takes a predetermined recommended measurement posture in a worn state of the belt being worn around the upper limb part, the transmission and reception unit faces both the artery running in the upper limb part and the heart. A transmission antenna unit included in the transmission and reception unit emits radio waves to each of the artery in the upper limb part and the heart. The reception antenna unit included in the transmission and reception unit receives the radio waves reflected by the artery in the upper limb part and/or a tissue being displaced in accordance with a pulse wave of the artery and by the heart and/or a tissue being displaced in accordance with a heartbeat of the heart. The vital sign detection unit acquires a pulse wave signal representing the pulse wave of the artery in the upper limb part and a heartbeat signal representing the heartbeat of the heart based on the outputs from the reception antenna unit.

In this manner, in this vital sign measurement device, the pulse wave signal representing a pulse wave of the artery in the upper limb part and the heartbeat signal representing the heartbeat of the heart are acquired simply with the living body physically wearing the belt wound around the upper limb part and taking the predetermined recommended measurement posture. Thus, for the measurement, no electrode needs to be mounted or attached to portions of the living body surrounding the heart. Furthermore, the recommended measurement posture taken by the living body may include a wide variety of postures such as a posture with the upper body erected or a lying posture, and thus a high degree of freedom is offered. Therefore, the vital sign measurement device imposes a small physical burden on the living body for the measurement.

In the vital sign measurement device of one embodiment,

the transmission antenna unit and the reception antenna unit are arranged along a plane in which the belt extends in a band form,

the transmission antenna unit includes:

a first transmission antenna that is provided on an inner circumferential surface side of the belt and emits the radio waves toward the artery in the upper limb part; and

a second transmission antenna that is provided on an outer circumferential surface side of the belt and emits the radio waves toward the heart, and

the reception antenna unit includes:

a first reception antenna that is disposed on the inner circumferential surface side of the belt and receives the radio waves reflected by the artery in the upper limb part and/or the tissue being displaced in accordance with the pulse wave of the artery; and

a second reception antenna that is disposed on the outer circumferential surface side of the belt and receives the radio waves reflected by the heart and/or the tissue being displaced in accordance with the heartbeat of the heart.

The “plane” in which the belt extends in a band shape refers to an inner circumferential surface facing the upper limb part in a worn state, or an outer circumferential surface opposite to the inner circumferential surface.

In the vital sign measurement device of this embodiment, on the inner circumferential surface side of the belt, the first transmission antenna emits radio waves toward the artery in the upper limb part, and the first reception antenna receives radio waves reflected by the artery and/or the tissue being displaced in accordance with the pulse wave of the artery. That is, the pulse wave of the artery in the upper limb part is detected by the first transmission antenna and the first reception antenna arranged on the inner circumferential surface side of the belt so as to face the upper limb part. Further, on the outer circumferential surface side of the belt, the second transmission antenna emits radio waves toward the heart, and the second reception antenna receives radio waves reflected from the heart and/or the tissue being displaced in accordance with the heartbeat of the heart. That is, the heartbeat of the heart is detected by the second transmission antenna and the second reception antenna arranged on the outer circumferential surface side of the belt so as to face the heart. With such a setting, the pulse wave signal and the heartbeat signal can be acquired with high accuracy.

The “transmission antenna” and the “reception antenna” may be provided separately from each other, but the present invention is not limited to this. An antenna element, which is a simple substance in terms of space, may be used as a transmission antenna and a reception antenna (that is, an antenna used for both transmission and reception) via a known circulator or the like.

In the vital sign measurement device of one embodiment, a shielding layer that shields the radio waves is provided between the first transmission antenna and the first reception antenna provided on the inner circumferential surface side of the belt and the second transmission antenna and the second reception antenna provided on the outer circumferential surface side of the belt.

In the vital sign measurement device according to the present embodiment, the shielding layer shields the radio waves between the first transmission antenna and the first reception antenna provided on the inner circumferential surface side of the belt and the second transmission antenna and the second reception antenna provided on the outer circumferential surface side of the belt. Thus, interference between the pulse wave signal and the heartbeat signal is suppressed. With such a setting, the pulse wave signal and the heartbeat signal can be acquired with higher accuracy.

In the vital sign measurement device of one embodiment, a frequency of the radio waves emitted toward the artery in the upper limb part and a frequency of the radio waves emitted toward the heart are different from each other.

With the vital sign measurement device of this embodiment, the radio waves reflected by the artery in the upper limb part and/or the tissue being displaced in accordance with the pulse wave of the artery, and the radio waves reflected by the heart and/or the tissue being displaced in accordance with the heartbeat of the heart can be distinguished from each other based on the frequencies so as not to interfere with each other. As a result, the pulse wave signal and the heartbeat signal can be acquired with higher accuracy.

In the vital sign measurement device of one embodiment,

the transmission antenna unit and the reception antenna unit are arranged along a plane in which the belt extends in a band form,

the transmission antenna unit includes a common third transmission antenna that is arranged along an inner circumferential surface side or an outer circumferential surface side of the belt or is embedded in the belt, and emits the radio waves toward both the artery in the upper limb part and the heart, and

the reception antenna unit includes:

a first reception antenna that is disposed on the inner circumferential surface side of the belt and receives the radio waves reflected by the artery in the upper limb part and/or the tissue being displaced in accordance with the pulse wave of the artery; and

a second reception antenna that is disposed on the outer circumferential surface side of the belt and receives the radio waves reflected by the heart and/or the tissue being displaced in accordance with the heartbeat of the heart.

Here, the third transmission antenna is “common” means that the third transmission antenna is configured as single antenna capable of simultaneously emitting radio waves to both the artery in the upper limb part and the heart. An example of such antenna is a dipole antenna. Emitting radio waves toward “both” includes cases where radio waves are emitted in all directions.

In the vital sign measurement device of this embodiment, the common third transmission antenna emits radio waves to both the artery in the upper limb part and the heart. On the inner circumferential surface side of the belt, the first reception antenna receives radio waves reflected by the artery in the upper limb part. Meanwhile, on the outer circumferential surface side of the belt, the second reception antenna receives radio waves reflected by the heart. In this vital sign measurement device, since the third transmission antenna is “common”, the configuration of the device can be simplified as compared with a case where two transmission antennas are provided, for example.

In the vital sign measurement device of one embodiment, a shielding layer that shields the radio waves is provided between the first reception antenna and the second reception antenna.

In the vital sign measurement device of this embodiment, the shielding layer shields radio waves between the first reception antenna and the second reception antenna. Thus, interference between the pulse wave signal and the heartbeat signal is suppressed. With such a setting, the pulse wave signal and the heartbeat signal can be acquired with high accuracy.

In the vital sign measurement device of one embodiment,

the third transmission antenna emits radio waves including a first frequency component and a second frequency component different from each other to both the artery in the upper limb part and the heart,

a component corresponding to the first frequency component in the radio waves reflected by the artery in the upper limb part and/or the tissue being displaced in accordance with the pulse wave of the artery is received through the first reception antenna, and

a component corresponding to the second frequency component in the radio waves reflected by the heart and/or the tissue being displaced in accordance with the heartbeat of the heart is received through the second reception antenna.

With the vital sign measurement device of this embodiment, a component corresponding to the first frequency component in the radio waves reflected by the artery in the upper limb part and/or the tissue being displaced in accordance with the pulse wave of the artery, and a component corresponding to the second frequency component in the radio waves reflected by the heart and/or the tissue being displaced in accordance with the heartbeat of the heart can be distinguished from each other based on the frequencies so as not to interfere with each other. As a result, the pulse wave signal and the heartbeat signal can be acquired with higher accuracy.

In the vital sign measurement device of one embodiment, the transmission antenna unit and the reception antenna unit are embedded in the belt so that the belt becomes flat on the inner circumferential surface side and the outer circumferential surface side of the belt.

In the vital sign measurement device of this embodiment, since the inner circumferential surface side of the belt is flat, the living body is free of uncomfortable feeling while wearing the belt (which may be felt if the belt has recesses and protrusions on the inner circumferential surface side). Furthermore, with the outer circumferential surface side of the belt is flat, the vital sign measurement device is less likely to break even when the outer circumferential surface of the belt comes into contact with a desk, a wall, or the like due to the activity of the living body. Furthermore, a better appearance can be achieved.

In the vital sign measurement device of one embodiment, a polarization direction of the radio waves transmitted from the first transmission antenna toward the artery in the upper limb part and a polarization direction of the radio waves emitted from the second transmission antenna toward the heart are different from each other.

With the vital sign measurement device of this embodiment, the radio waves reflected by the artery in the upper limb part and/or the tissue being displaced in accordance with the pulse wave of the artery, and the radio waves reflected by the heart and/or the tissue being displaced in accordance with the heartbeat of the heart can be distinguished from each other based on polarization direction so as not to interfere with each other. As a result, the pulse wave signal and the heartbeat signal can be acquired with higher accuracy as a result.

The polarization directions of the radio waves can be set different between the first transmission antenna and the second transmission antenna in various ways. For example, the first transmission antenna and the second transmission antenna may each be formed by patch antenna with a rectangular pattern shape, and the position of the feed point may be set to be different from each other between the patch antennas.

In the vital sign measurement device of one embodiment,

a portion of the belt corresponding to the transmission and reception unit is provided with

a transmission circuit that supplies power for the transmission antenna unit to emit the radio waves, and

a reception circuit that at least amplifies a signal received by the reception antenna unit.

In the vital sign measurement device of this embodiment, a power feeding path from the transmission circuit to the transmission antenna unit can be made relatively short, whereby the deterioration of the waveform of the radio wave can be suppressed. Furthermore, a reception path from the reception antenna unit to the reception circuit can be made relatively short. As a result, the pulse wave signal and the heartbeat signal can be acquired with higher accuracy.

In another aspect, a blood pressure measurement device of the present disclosure is a blood pressure measurement device that measures blood pressure of a living body, the blood pressure measurement device comprising:

the above vital sign measurement device;

a time difference acquisition unit that acquires as a pulse transit time, a time difference between the pulse wave signal and the heartbeat signal acquired by the vital sign detection unit; and

a first blood pressure calculation unit that calculates a blood pressure value based on the pulse transit time acquired by the time difference acquisition unit by using a predetermined correspondence formula between the pulse transit time and the blood pressure.

In the blood pressure measurement device of the present disclosure, the time difference acquisition unit acquires the time difference between the pulse wave signal and the heartbeat signal acquired by the vital sign detection unit, as a pulse transit time (PTT). The first blood pressure calculation unit calculates a blood pressure value based on the pulse transit time acquired by the time difference acquisition unit using a predetermined correspondence formula between the pulse transit time and the blood pressure. Thus, with this blood pressure measurement device, a blood pressure value can be obtained.

In the blood pressure measurement device of one embodiment, the vital sign detection unit, the time difference acquisition unit, and the first blood pressure calculation unit are integrally provided to the belt.

In the blood pressure measurement device of this embodiment, unlike in a case where vital sign detection unit, the time difference acquisition unit, and the first blood pressure calculation unit are provided to be outside of and separated from the belt, no wiring needs to extend to the outside of the belt to obtain the pulse wave signal, the heartbeat signal, the PTT, and the blood pressure value from the output of the reception antenna unit. Thus, with the blood pressure measurement device, the living body needs not be bothered by the wiring cable at the time of the measurement, and thus the physical load is small.

In the blood pressure measurement device of one embodiment,

a fluid bag for pressurizing the upper limb part is attached to the belt,

the blood pressure measurement device comprises:

a pressure control unit that supplies air to the fluid bag to control pressure; and

a second blood pressure calculation unit that calculates a blood pressure through an oscillometric method based on the pressure in the fluid bag, and

the pressure control unit and the second blood pressure calculation unit are integrally provided to the belt, or are provided to a main body integrally provided to the belt.

In the blood pressure measurement device according to this embodiment, blood pressure measurement (estimation) based on the PTT and blood pressure measurement by the oscillometric method can be performed using the same belt. Thus, usability for the subject as the living body can be improved. In addition, the PTT method (blood pressure measurement based on PTT) enabling continuous measurement but with low accuracy may be performed to capture sharp blood pressure rise, and using the sharp blood pressure rise as a trigger, more accurate measurement through the oscillometric method can be started.

In another aspect, an apparatus of the present disclosure is an apparatus comprising the above vital sign measurement device or the above blood pressure measurement device.

The apparatus of the present disclosure may include the above vital sign measurement device or the above blood pressure measurement device, and may include a functional unit that performs a further function. With this apparatus, a pulse wave signal representing a pulse wave of an artery in an upper limb part of a living body and a heartbeat signal representing a heartbeat of the heart can be obtained, or a blood pressure value can be calculated (estimated). This apparatus can perform various further functions.

In another aspect, a vital sign measurement method of the present disclosure is a vital sign measurement method that measures a pulse wave of an artery and a heartbeat of a heart of a living body by using the above vital sign measurement device, the vital sign measurement method comprising:

wearing the belt around the upper limb part; and

causing the transmission and reception unit to face both an artery running in the upper limb part and the heart by the living body taking a predetermined posture in a worn state of the belt being worn around the upper limb part;

emitting radio waves to each of the artery in the upper limb part and the heart through the transmission antenna unit;

receiving radio waves reflected by the artery in the upper limb part and/or a tissue being displaced in accordance with a pulse wave of the artery and by the heart and/or a tissue being displaced in accordance with the heartbeat of the heart through the reception antenna unit; and

acquiring, by the vital sign detection unit, a pulse wave signal representing the pulse wave of the artery in the upper limb part and a heartbeat signal representing the heartbeat of the heart based on an output from the reception antenna unit.

In this vital sign measurement method, the pulse wave signal representing a pulse wave of the artery in the upper limb part and the heartbeat signal representing the heartbeat of the heart are acquired under a simple physical condition in which the living body wears the belt around the upper limb part and takes the predetermined recommended measurement posture. Thus, for the measurement, no electrode needs to be mounted or attached to portions of the living body surrounding the heart. Furthermore, the recommended measurement posture taken by the living body may include a wide variety of postures such as a posture with the upper body erected or a lying posture, and thus a high degree of freedom is offered. Therefore, the physical burden on the living body for measurement is small.

In another aspect, a blood pressure measurement method of the present disclosure is a blood pressure measurement method that measures blood pressure of a living body, the blood pressure measurement method comprising:

acquiring a pulse wave signal representing the pulse wave of the artery in the upper limb part and a heartbeat signal representing the heartbeat of the heart by executing the above vital sign measurement method;

acquiring, as a pulse transit time, a time difference between the pulse wave signal and the heartbeat signal; and

calculating a blood pressure value based on the acquired pulse transit time by using a predetermined correspondence formula between the pulse transit time and the blood pressure.

With the blood pressure measurement method according to the present disclosure, a blood pressure value is acquired under a simple physical condition where a living body wears a belt around the upper limb part and taking a predetermined recommended measurement posture. Therefore, the physical burden on the living body for measurement is small.

As is clear from the above description, the vital sign measurement device according to the present disclosure imposes a small physical burden on the living body for the measurement. Furthermore, the blood pressure measurement device, the vital sign measurement method, and the blood pressure measurement method according to the present disclosure impose a small physical burden on the living body for the measurement. Further, with the apparatus of the present disclosure, various functions can be executed in addition to the acquisition of the pulse wave signal and the heartbeat signal, or the calculation of the blood pressure value.

The above embodiments are merely examples, and various modifications can be made without departing from the scope of the present invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A vital sign measurement device that measures a pulse wave of an artery and a heartbeat of a heart of a living body, the vital sign measurement device comprising:
a belt to be worn around an upper limb part of the living body; and
a transmission and reception unit that is capable of transmitting and receiving radio waves, the transmission and reception unit being provided at a portion of the belt to face both an artery running in the upper limb part and the heart when the living body takes a predetermined recommended measurement posture in a worn state of the belt being worn around the upper limb part, wherein
the transmission and reception unit includes:
a transmission antenna unit that emits radio waves to each of the artery in the upper limb part and the heart; and
a reception antenna unit that receives radio waves reflected by the artery in the upper limb part and/or a tissue being displaced in accordance with a pulse wave of the artery and by the heart and/or a tissue being displaced in accordance with the heartbeat of the heart, and
the vital sign measurement device further comprises a vital sign detection unit that acquires a pulse wave signal representing the pulse wave of the artery in the upper limb part and a heartbeat signal representing the heartbeat of the heart based on an output from the reception antenna unit.

2. The vital sign measurement device according to claim 1, wherein
the transmission antenna unit and the reception antenna unit are arranged along a plane in which the belt extends in a band form,
the transmission antenna unit includes:
a first transmission antenna that is provided on an inner circumferential surface side of the belt and emits the radio waves toward the artery in the upper limb part; and
a second transmission antenna that is provided on an outer circumferential surface side of the belt and emits the radio waves toward the heart, and
the reception antenna unit includes:
a first reception antenna that is disposed on the inner circumferential surface side of the belt and receives the radio waves reflected by the artery in the upper limb part and/or the tissue being displaced in accordance with the pulse wave of the artery; and
a second reception antenna that is disposed on the outer circumferential surface side of the belt and receives the radio waves reflected by the heart and/or the tissue being displaced in accordance with the heartbeat of the heart.

3. The vital sign measurement device according to claim 2, wherein a shielding layer that shields the radio waves is provided between the first transmission antenna and the first reception antenna provided on the inner circumferential surface side of the belt and the second transmission antenna and the second reception antenna provided on the outer circumferential surface side of the belt.

4. The vital sign measurement device according to claim 1, wherein a frequency of the radio waves emitted toward the artery in the upper limb part and a frequency of the radio waves emitted toward the heart are different from each other.

5. The vital sign measurement device according to claim 1, wherein
the transmission antenna unit and the reception antenna unit are arranged along a plane in which the belt extends in a band form,
the transmission antenna unit includes a common third transmission antenna that is arranged along an inner circumferential surface side or an outer circumferential surface side of the belt or is embedded in the belt, and emits the radio waves toward both the artery in the upper limb part and the heart, and
the reception antenna unit includes:
a first reception antenna that is disposed on the inner circumferential surface side of the belt and receives the radio waves reflected by the artery in the upper limb part and/or the tissue being displaced in accordance with the pulse wave of the artery; and
a second reception antenna that is disposed on the outer circumferential surface side of the belt and receives the radio waves reflected by the heart and/or the tissue being displaced in accordance with the heartbeat of the heart.

6. The vital sign measurement device according to claim 5, wherein a shielding layer that shields the radio waves is provided between the first reception antenna and the second reception antenna.

7. The vital sign measurement device according to claim 5, wherein the third transmission antenna emits radio waves including a first frequency component and a second frequency component different from each other to both the artery in the upper limb part and the heart,
a component corresponding to the first frequency component in the radio waves reflected by the artery in the upper limb part and/or the tissue being displaced in accordance with the pulse wave of the artery is received through the first reception antenna, and
a component corresponding to the second frequency component in the radio waves reflected by the heart and/or the tissue being displaced in accordance with the heartbeat of the heart is received through the second reception antenna.

8. The vital sign measurement device according to claim 1, wherein the transmission antenna unit and the reception antenna unit are embedded in the belt so that the belt becomes flat on the inner circumferential surface side and the outer circumferential surface side of the belt.

9. The vital sign measurement device according to claim 2, wherein a polarization direction of the radio waves transmitted from the first transmission antenna toward the artery in the upper limb part and a polarization direction of the radio waves emitted from the second transmission antenna toward the heart are different from each other.

10. The vital sign measurement device according to claim 1, wherein
a portion of the belt corresponding to the transmission and reception unit is provided with
a transmission circuit that supplies power for the transmission antenna unit to emit the radio waves, and
a reception circuit that at least amplifies a signal received by the reception antenna unit.

11. A blood pressure measurement device that measures blood pressure of a living body, the blood pressure measurement device comprising:
the vital sign measurement device according to claim 1;
a time difference acquisition unit that acquires as a pulse transit time, a time difference between the pulse wave signal and the heartbeat signal acquired by the vital sign detection unit; and
a first blood pressure calculation unit that calculates a blood pressure value based on the pulse transit time acquired by the time difference acquisition unit by using a predetermined correspondence formula between the pulse transit time and the blood pressure.

12. The blood pressure measurement device according to claim 11, wherein the vital sign detection unit, the time difference acquisition unit, and the first blood pressure calculation unit are integrally provided to the belt.

13. The blood pressure measurement device according to claim 11, wherein
a fluid bag for pressurizing the upper limb part is attached to the belt,
the blood pressure measurement device comprises:
a pressure control unit that supplies air to the fluid bag to control pressure; and
a second blood pressure calculation unit that calculates a blood pressure through an oscillometric method based on the pressure in the fluid bag, and
the pressure control unit and the second blood pressure calculation unit are integrally provided to the belt, or are provided to a main body integrally provided to the belt.

14. An apparatus comprising the vital sign measurement device according to claim 1.

15. A vital sign measurement method that measures a pulse wave of an artery and a heartbeat of a heart of a living body by using the vital sign measurement device according to claim 1, the vital sign measurement method comprising:
wearing the belt around the upper limb part; and
causing the transmission and reception unit to face both an artery running in the upper limb part and the heart by the living body taking a predetermined posture in a worn state of the belt being worn around the upper limb part;
emitting radio waves to each of the artery in the upper limb part and the heart through the transmission antenna unit;
receiving radio waves reflected by the artery in the upper limb part and/or a tissue being displaced in accordance with a pulse wave of the artery and by the heart and/or a tissue being displaced in accordance with the heartbeat of the heart through the reception antenna unit; and
acquiring, by the vital sign detection unit, a pulse wave signal representing the pulse wave of the artery in the upper limb part and a heartbeat signal representing the heartbeat of the heart based on an output from the reception antenna unit.

16. A blood pressure measurement method that measures blood pressure of a living body, the blood pressure measurement method comprising:
acquiring a pulse wave signal representing the pulse wave of the artery in the upper limb part and a heartbeat signal representing the heartbeat of the heart by executing the vital sign measurement method according to claim 15;
acquiring, as a pulse transit time, a time difference between the pulse wave signal and the heartbeat signal; and
calculating a blood pressure value based on the acquired pulse transit time by using a predetermined correspondence formula between the pulse transit time and the blood pressure.
